(19) United States
(12) Patent Application Publication
(10) Pub. No.: US 2022/0178867 A1
(43) Pub. Date: Jun. 9, 2022

(54) SYSTEMS, DEVICES AND METHODS FOR SENSING BIOMARKERS USING ENZYMATIC AND IMMUNOSENSING ELECTROCHEMICAL DETECTION TECHNIQUES

(71) Applicant: The Regents of the University of California, Oakland, CA (US)

(72) Inventors: Joseph Wang, La Jolla, CA (US); Farshad Tehrani, La Jolla, CA (US); Eva Vargas Orgaz, La Jolla, CA (US); Hazhir Teymourian, La Jolla, CA (US); Eyal Dassau, Cambridge, MA (US)

(21) Appl. No.: 17/438,356

(22) PCT Filed: Mar. 12, 2020
(86) PCT No.: PCT/US2020/022489

§ 371 Date: Sep. 10, 2021

Related U.S. Application Data
(60) Provisional application No. 62/817,380, filed on Mar. 12, 2019.

(51) Int. Cl. G01N 27/327; C12Q 1/26; C12Q 1/00; G01N 33/49

(52) U.S. Cl. CPC G01N 27/3275; C12Q 1/26; G01N 27/3271; G01N 2800/042; G01N 33/492; G01N 2333/62; C12Q 1/006

(57) ABSTRACT

Disclosed are devices, systems and methods for monitoring one or more biomarkers using an electrochemical immunosensor sensor with an integrated enzymatic and immunosensing electrochemical detection capability. In some aspects, an electrochemical sensor device for monitoring glucose and insulin includes a substrate; and a plurality of electrodes disposed on the substrate, the plurality of electrodes including a first electrode to sense glucose, a second electrode to sense insulin, and a counter electrode to the first and second electrodes, in which the first electrode includes a glucose oxidase enzyme linked to a surface of the first electrode, and the second electrode includes an insulin capture antibody linked to a second electrode through a self-assembly monolayer, and in which, when the device is electrically coupled to an electronics unit, the device is operable to detect insulin and glucose from a fluid.

### CROSS-REFERENCE TO RELATED APPLICATIONS

This patent document claims priorities to and benefits of U.S. Provisional Patent Application No. 62/817,380, titled “SYSTEMS, DEVICES AND METHODS FOR SENSING BIOMARKERS USING ENZYMATIC AND IMMUNOSENSING” and filed on Mar. 12, 2019. The entire content of the aforementioned patent application is incorporated by reference as part of the disclosure of this patent document.

### TECHNICAL FIELD

This patent document relates to systems, devices, and processes that use biosensing for analyte detection.

### BACKGROUND

Biosensors can provide real-time detection of physiological substances and processes in living things. A biosensor is an analytical tool that can detect a chemical, substance, or organism using a biologically sensitive component coupled with a transducing element to convert a detection event into a signal for processing and/or display. Biosensors can use biological materials as the biologically sensitive component, e.g., such as biomolecules including enzymes, antibodies, nucleic acids, etc., as well as living cells. For example, molecular biosensors can be configured to use specific chemical properties or molecular recognition mechanisms to identify target agents, which can be useful in diagnosis and treatments for various health care applications.

Diabetes Mellitus (DM), a series of chronic metabolic diseases characterized by inadequate glucose metabolism, is a worldwide epidemic, involving nearly 30 million people in the U.S., and costing nearly $250 billion in direct and non-direct health costs. According to the American Diabetes Association, by the year 2034 the number of diagnosed and undiagnosed people with diabetes will increase from 23.7 million to 44.1 million. Despite the considerable amount of research focused on the diagnosis and management of various diabetic disorders, there is still no cure for diabetes.

### SUMMARY

Disclosed are devices, systems and methods for monitoring multiple analytes using a single, integrated electrochemical immunosensor sensing platform for enzymatic or direct electrochemical detection and immunosensing detection capability. Also disclosed are cost-effective, scalable methods for manufacturing such multi-analyte, multi-detection modality sensor devices.

In some embodiments, a multi-modal, multi-analyte sensor chip includes an immunosensing contingent and electrochemical sensing contingent configured on a single substrate, where the “ImmunoChip” strip can provides decentralized insulin detection in biofluids and, when integrated with the electrochemical sensing contingent, can provide a dual-analyte glucose/insulin (G/I) chip that simultaneously detects these diabetes markers in a single microliter droplet of a body fluid. The disclosed multi-analyte sensor chips can be fabricated by a newly-developed masking/sputtering method, which permits the cost-effective and scalable fabrication of the sensor chips.

Implementations of example embodiments of the sensor chip demonstrates a single fully-integrated chip for simultaneous and fast on-the-spot detection of multiple diabetic biomarkers including, but not limited to, insulin and glucose. In some embodiments, the sensor chip incorporates multiple immunoassay and enzymatic detection systems in a single unit. In some embodiments, the sensor chip is fabricated using a method that includes chip units, which is extremely low-cost and highly scalable. In some embodiments, the sensor chip is adoptable to a wearable and continuous monitoring platform, in which each target analyte can be continuously detected by the sensor chip in a single on-body form.

In some aspects, a sensor device for simultaneous monitoring two or more analytes includes a substrate; and two or more detecting electrodes disposed on the substrate, the two or more detecting electrodes including a first electrode to sense a first analyte by an enzymatic or direct electrochemical detection, and a second electrode to sense a second analyte by an immunosensing detection; a first functionalization layer disposed on the first electrode, the first functionalization layer including a catalyst to facilitate an electrochemical reaction to detect the first analyte at the first electrode; a second functionalization layer disposed on the second electrode, the second functionalization layer including a capture antibody to facilitate an electrochemical immune assay reaction to detect the second analyte at the second electrode; and a counter electrode to detect a signal with respect to that detected by the one or both of the first electrode and the second electrode.

In some aspects, a method for simultaneously detecting multiple analytes on a single sensor platform includes contacting a fluid sample with a sensor device comprising a substrate and a plurality of electrodes disposed on the substrate, the electrodes including a first electrode for sensing a first analyte, a second electrode for sensing a second analyte, and a counter electrode; and measuring a parameter associated with the first analyte or the second analyte, or both.

The subject matter described in this patent document can be implemented in specific ways that provide one or more of the following features.

### BRIEF DESCRIPTION OF THE DRAWINGS

FIG. 1A shows a diagram of an example embodiment of a multi-analyte, multi-detection modality sensor device in accordance with the present technology.

FIG. 1B shows a block diagram of an example embodiment of an electronic device that can interface with various embodiments of the sensor device of FIG. 1A.

FIG. 1C shows illustrative diagrams of example embodiments of immunosensor sensors and integrated electrochemical immunosensor sensors in accordance with the present technology, with an enzymatic and immunosensing electrochemical detection capability of glucose and insulin.

FIG. 1D shows a diagram illustrating an example implementation of a dual biomarker integrated electrochemical immunosensor sensor chip device shown in FIG. 1C.

FIG. 2 shows images of different arrays of fabricated sensor chips in accordance with various embodiments of the sensor devices shown in FIG. 1C.

FIG. 3 shows illustrations and data plots depicting example results using example embodiments of a glucose/insulin sensor in accordance with the present technology.

FIG. 4 shows an exemplary method for fabricating a multi-biomarker sensor chip device in accordance with the present technology.

FIG. 5 shows an exemplary protocol for fabricating an example embodiment of a glucose/insulin sensor device in accordance with the present technology.

FIGS. 6A and 6B show exemplary plots depicting the amperometric responses of an example glucose/insulin sensor.

FIG. 6C shows an exemplary cyclic voltammogram plot of an example glucose/insulin sensor.

FIG. 7 shows exemplary plots of the amperometric responses measured for an example glucose/insulin sensor comparing one step vs. two step incubation protocols regarding the insulin immunoassay to confirm cross-talk between the glucose and insulin sensors.

FIG. 8A shows a panel of images of an example array of glucose/insulin sensor chip devices, on a single polyethylene terephthalate (PET) substrate, in accordance with the present technology.

FIGS. 8B and 8C show illustrative diagrams depicting production of detecting electrodes of a multi-biomarker sensor chip for the localized detection of glucose and insulin on a single sensor chip in accordance with the present technology.

FIG. 8D shows an exemplary scheme for the measurement of glucose and insulin from a patient's blood or saliva using the glucose/insulin sensor in accordance with the present technology.

FIGS. 9A-9G show exemplary plots (in FIGS. 9A-9F) evaluating the performance of the electrochemical- and immune-sensing capability of an example glucose/insulin sensor device (illustrated in FIG. 9G).

FIGS. 10A-10F show exemplary plots for evaluating the cross-talk of an example glucose/insulin sensor with glucose and insulin mixture solutions in example implementations of the glucose/insulin sensor.

FIGS. 10I-10L show data plots evaluating the performance of example electrochemical sensing of an example embodiment of the glucose/insulin sensor chip using blood samples (FIGS. 10K, 10L) and saliva samples (FIGS. 10I, 10J).

FIGS. 11A-11C show diagrams illustrating an example embodiment of a dual biomarker sensor chip device for the simultaneous detection of cortisol and insulin biomarkers in accordance with the present technology.

FIG. 11D shows a diagram illustrating an example embodiment of a method for fabricating the example dual biomarker sensor chip shown in FIGS. 11A-11C.

FIGS. 12A-12J shows illustrations and data plots depicting an example implementation for evaluating cross-talk during electrochemical transduction for an example cortisol-insulin sensor chip device.

FIGS. 12K and 12L show data plots of example results for evaluation of a buffer employed in an electrochemical transduction using the example cortisol-insulin sensor chip device.

FIGS. 13A and 13B show data plots depicting example results from evaluation of the experimental conditions for ALP-based electrochemical transduction.

FIGS. 13C and 13D show data plots depicting example results for an example optimization of experimental variables affecting immunoassay performance.

FIGS. 13E, 13F and 13G show data plots depicting example results of an evaluation of the effect of the buffer pH on the amperometric responses.

FIG. 14 shows a data plot and a diagram depicting example results of an evaluation of an example sequential transduction protocol for the example dual C/I sensor chip, with the example protocol described in the table of the diagram.

FIGS. 15A-15E show illustrative diagrams and data plots depicting example results of an example implementation for evaluating electrochemical sensing performance of example C/I dual chip devices in phosphate buffer solution.

FIGS. 16A-16C show data plots and accompanying illustrations depicting the electrochemical sensing performance of example C/I dual chips in non-treated human serum.

### DETAILED DESCRIPTION

Historically, the monitoring of diabetes has relied solely on blood glucose detection by extracting blood through finger pricking and the use of disposable glucose meter test strips. However, currently there is a growing interest on the importance of insulin, a polypeptide hormone that is vital for glucose metabolism, in medical problems related to diabetes and some other diseases. For better management of diabetes, rapid detection of pancreatic islet-cell malfunction, clear definition of hypoglycemia, precise diagnosis of insulinoma, and early prediction of trauma, there is an urgent need to develop diagnostic devices to monitor levels of insulin and glucose in a simultaneous manner. However, due to the pico molar (pM) levels of insulin in blood, development of highly sensitive sensors are essential. The current insulin detection methods, such as ELISA, RIA, and CLIA, are not amenable to easy use a practitioner or patient.

Disclosed are devices, systems and methods for monitoring multiple analytes using a single, integrated electrochemical immunosensor sensing platform for enzymatic or direct electrochemical detection and immunosensing detection capability. Also disclosed are cost-effective, scalable methods for manufacturing such multi-analyte, multi-detection modality sensor devices.

The disclosed devices, systems and methods can provide on-the-spot, decentralized monitoring of biomarkers relevant to diabetes found in physiological ranges of human blood, saliva, and interstitial fluid samples using a single, disposable, compact, patient- and practitioner-friendly electrochemical sensor. In some implementations, the integrated electrochemical immunosensor can detect any disease biomarker of relevance in clinical practice near the patient and in point-of-care settings. In particular, in some embodiments, the sensor can be configured as an electrochemical chip for point-of-care monitoring of multiple diabetes relevant biomarkers found within the physiological ranges of human blood, saliva, and interstitial fluid (ISF) samples, e.g., enabling detection of any disease biomarker of relevance for screening. The sensor can be configured as a streamlined immunosensor (e.g., sometimes referred to as the “ImmunoChip strip”) for decentralized detection of a particular analyte, such as insulin, in a biofluid of a patient; and the sensor can be configured as a dual- or multi-analyte electrochemical immunosensor on a single chip for simultaneously detecting two (or more) analytes, such as insulin and glucose in the patient's biofluid, e.g., such as in a single microliter droplet of a body fluid for detecting these diabetes biomarkers.

A major goal in disease screening, diagnosis and control has been the development of bioassay platforms capable of simultaneous measurements of different types of analytes in a single assay. Major advances toward multiplexed biomarker detection chips based on either immunoassay or enzymatic bioassays have thus been reported. However, the combination of both enzymatic and immunoassay sensing systems—each with its own fundamentally distinct sensing format and fabrication requirements—into a single disposable chip platform is yet to be addressed.

There are currently no reports of a single chip strip capable of on-the-spot simultaneous detection of insulin and glucose in the same microliter biofluid sample. Similarly, no user-friendly chip strip is available for decentralized testing of insulin. Different methodologies have been proposed for the detection of insulin. Conventional antigen-antibody-based methods to quantify insulin concentrations, such as the radioimmunoassay (MA) and enzyme-linked immunosorbent assay (ELISA), are extremely tedious, laborious and long-lasting, and require expensive instruments and qualified staff.

In contrast with conventional approaches, an electrochemical immunosensor chip in accordance with some embodiments of the present technology provides high sensitivity and selectivity, precision and accuracy, relatively low cost, minimum sample requirement, simplicity of operation and possible integration in compact analytical devices. For example, the state-of-the-art ELISA immunoassay used for insulin measurement, require a long time for analysis (e.g., >1 hour) and large volumes of patient's blood or saliva sample (e.g., 50-100 as well as have to be performed in centralized laboratories, increasing the cost of the analysis. However, the electrochemical immunosensor chip of the present technology provides low-cost disposable strips and allows detection of trace insulin levels within low volumes (e.g., <10 μL) of blood and/or saliva samples in less than 20 minutes, all of which provides an unprecedented personal point-of-care diagnostics tool to the patients.

In some example embodiments, a dual marker biosensor device capable of integrating enzymatic and immunosensing electrochemical detection protocols into a single electrochemical immunosensor chip is disclosed for detecting glucose and insulin, referred to as the G/I sensor chip. Implementations of the G/I sensor chip demonstrate reliable decentralized detection of insulin (I) and glucose (G), as two key diabetes biomarkers.

Point-of-care tandem measurements of glucose and insulin in the same microliter sample droplet is of tremendous importance to personalized diabetes management towards improved estimates of insulin sensitivity and enhanced regulation of glucose levels. Despite major advances in diabetes management, including self-testing glucose strips, the ability to rapidly measure insulin and to concurrently monitor both glucose and insulin represents an unmet challenge that if realized, can transform clinical care.

The integration of the fundamentally different enzymatic and immunochemical assays onto a single chip device for the simultaneous detection of insulin and glucose, faces major fabrication, operational and sensing challenges, all of which have been addressed successfully in the various embodiments of the present disclosure. These challenges can include (i) the huge difference between the physiological concentrations of insulin (pM) and glucose (mM); (ii) the different surface chemistries required for enzymatic and immunosensors; (iii) the completely different assay formats and time scales; (iv) the need for compact low-cost chip footprint capable of simultaneous bioassays of microliter (e.g., ˜10 μL) samples; (v) ultra low (e.g., picometer (pM)) insulin concentration in bodily fluids which requires ultrasensitive immunoassay format; (vi) potential cross-talk and cross-reactivity between the neighboring biocatalytic and bioaffinity sensors; and (viii) short analysis time to prevent changes in biomarker levels, avoid surface biofouling and provide timely information.

The electrochemical immunosensor chip of the present technology can provide inherent high sensitivity and selectivity, precision and accuracy, relatively low cost, minimum sample requirement, simplicity of operation and possible integration in compact analytical devices.

While several of the disclosed embodiments of the multi-marker biosensor technology described herein are primarily directed to detection of glucose and insulin, it is understood these analytes are used in examples that facilitate understanding of the underlying concepts, and that the disclosed embodiments can also include simultaneous monitoring and detection of other analytes.

For example, the electrochemical chip can be expanded for additional disease biomarkers, particularly using electrochemical assay of ketone bodies (through enzymatic detection of hydroxybutyrate) and competitive immunoassays of cortisol, along with glucose and insulin. The electrode array thus allows simultaneous measurements of four biomarkers with no cross talk in a single drop of the fluid. Dual biomarkers detection, such as simultaneous immunoassays of insulin and cortisol and enzymatic assays of glucose and hydroxybutyrate can also be carried out.

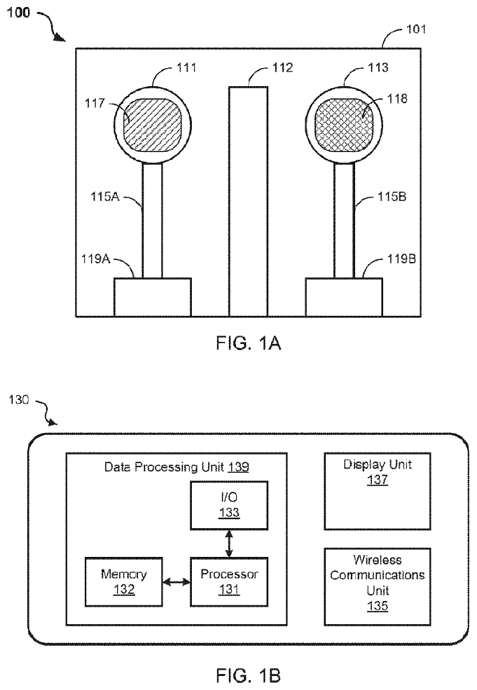

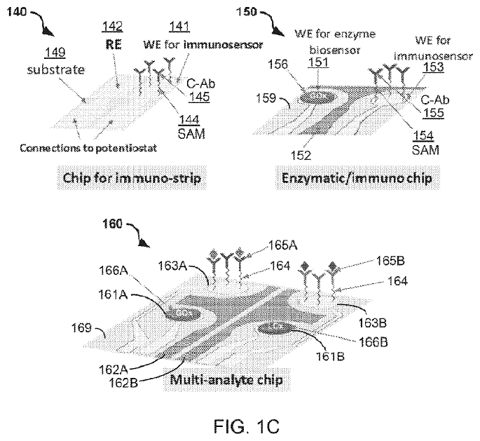

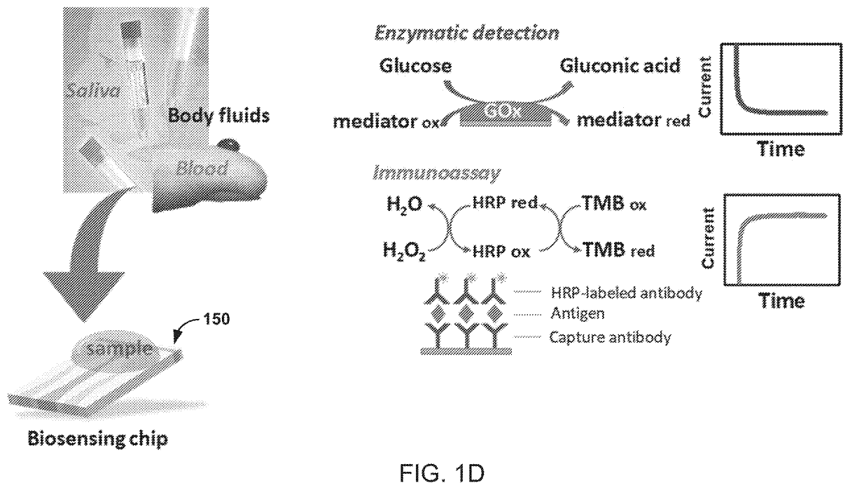

FIG. 1A shows a diagram of an example embodiment of a multi-analyte, multi-detection modality sensor device 100 in accordance with the present technology. The sensor device 100 includes a substrate 101 and at least two electrodes 111, 112 disposed on the substrate 101. The substrate 101 includes an electrically insulative material, such as a plastic material (e.g., polyethylene terephthalate (PET), polyethylene terephthalate glycol (PETG), polyethylene naphthalate (PEN), polyimide (PI), or other); and the electrodes 111, 112 include an electrically conductive material, such as a metal (e.g., gold). In the example embodiment shown in the diagram, the sensor device 100 includes two detecting electrodes 111 and 113 that can be disposed on opposing sides of the electrode 112 serving as a counter/reference electrode. In other example embodiments, the detecting electrodes 111, 113 can be configured proximate the same side of the counter/reference electrode 112. In some implementations of the sensor device 100, the detecting electrode 111 is configured to sense an analyte by an enzymatic or direct electrochemical detection, and the detecting electrode 113 is configured to sense the analyte or a different analyte by an immunosensing detection. The counter/reference electrode 112 is configured to detect a signal with respect to that detected by the one or both of the first electrode and the second electrode.

In some embodiments, the multi-analyte, multi-detection modality sensor device 100 can be configured for single analyte detection, where the sensor device 100 includes a single detecting electrode (e.g., the electrode 111) and a single reference/counter electrode (e.g., electrode 112). For example, the sensor device 100 can be employed as an amperometric sensor to measure the electrical current response (at a fixed potential) for detecting the concentration of an analyte in a fluid on the sensor device 100, where an electroactive species is produced, consumed or changed as the target analyte (e.g., enzyme) interacts with a reaction material (e.g., a catalyst, an antibody, or other molecular probe) in a functionalization layer 117 (also referred to as sensing layer 117) disposed on the detecting electrode 111. In some embodiments, the sensor device 100 can include a second sensing layer 118 disposed on the detecting electrode 113. In such embodiments, the sensor device 100 can be implemented to simultaneously detect a first target analyte at the detecting electrode 111 and a second target analyte at the detecting electrode 113 via an electrochemical sensing technique operated using both detecting electrodes with respect to the counter/reference electrode 112 or by different electrochemical sensing techniques operated concurrently and/or intermittently on each of the detecting electrodes with respect to the counter/reference electrode 112.

In some embodiments, the sensor device 100 includes a conduit 115 and an interface pad 119, where the conduit 115 is positioned between a detecting electrode and the interface pad 119 to electrically connect the detecting electrode to the interface pad 119. In the example shown, a conduit 115A is disposed on or within the substrate 101 between and coupled to the detecting electrode 111 and an interface pad 119A; and conduit 115B is disposed on or within the substrate 101 between and coupled to the detecting electrode 113 and an interface pad 119B.

FIG. 1B shows a block diagram of an example embodiment of an electronic device 130 that can electrically interface to the sensor device 100 for various implementations. For example, the electronic device 130 can connect an electrical circuit of the electronic device 130 with the interface pads 119A, 119B of the sensor device 100. In various implementations, the electronic device 130 is operable to store and execute software applications to implement various sensing protocol algorithms and/or implement various functionalities of the sensor device 100. In various implementations, the electronic device 130 can be implemented as a portable signal processing and/or computing device, which can include a mobile communications device, such as a smartphone, tablet or wearable device, like a smartwatch, glasses, etc.; and/or the electronic device 130 can be implemented as a stationary signal processing and/or computing device, such as a desktop computer and amplifier. In some embodiments, the electronic device 130 includes a dongle that couples to the interface pad 119 of the sensor device 100 to wirelessly connect to computing components (e.g., a data processing unit) of the electronic device 130.

In some embodiments, the electronic device 130 includes a data processing unit 139 includes a processor 131 to process data, a memory 132 in communication with the processor 131 to store data, and an input/output unit (I/O) 133 to interface the processor 131 and/or memory 132 to other modules, units or devices, including other external computing devices. For example, the processor 131 can include a central processing unit (CPU) or a microcontroller unit (MCU). For example, the memory 132 can include and store processor-executable code, which when executed by the processor, configures the data processing unit 139 to perform various operations, e.g., such as receiving information, commands, and/or data, processing information and data, and transmitting or providing information/data to another device. In some implementations, the data processing unit 139 can transmit raw or processed data to a computer system or communication network accessible via the Internet (referred to as ‘the cloud’) that includes one or more remote computational processing devices (e.g., servers in the cloud). To support various functions of the data processing unit 139, the memory 132 can store information and data, such as instructions, software, values, images, and other data processed or referenced by the processor. For example, various types of Random Access Memory (RAM) devices, Read Only Memory (ROM) devices, Flash Memory devices, and other suitable storage media can be used to implement storage functions of the memory 132. In some embodiments, the data processing unit 139 includes a wireless communication unit 135, such as a wireless transmitter to transmit stored and/or processed data or a wireless transceiver (Tx/Rx) to transmit and receive data. The I/O 133 of the data processing unit 139 can interface the data processing unit 139 with the wireless communications unit 135 to utilize various types of wired or wireless interfaces compatible with typical data communication standards, for example, which can be used in communications of the data processing unit 139 with other devices, via a wireless transmitter/receiver (Tx/Rx) unit, e.g., including, but not limited to, Bluetooth, Bluetooth low energy, Zigbee, IEEE 802.11, Wireless Local Area Network (WLAN), Wireless Personal Area Network (WPAN), Wireless Wide Area Network (WWAN), WiMAX, IEEE 802.16 (Worldwide Interoperability for Microwave Access (WiMAX)), 3G/4G/LTE/5G cellular communication methods, NFC (Near Field Communication), and parallel interfaces. In some embodiments, the data processing unit 139 includes a display unit 137, which can include a visual display such as a display screen, an audio display such as a speaker, or other type of display or combinations thereof. The I/O 133 of the data processing unit 139 can also interface with other external interfaces, sources of data storage, and/or visual or audio display devices, etc. to retrieve and transfer data and information that can be processed by the processor 131, stored in the memory 132, or exhibited on an output unit (e.g., display unit 137) of the electronic device 500 or an external device. For example, the display unit 137 can be configured to be in data communication with the data processing unit 139, e.g., via the I/O 133, to provide a visual display, an audio display, and/or other sensory display that produces the user interface of the software application. In some examples, the display unit 137 can include various types of screen displays, speakers, or printing interfaces, e.g., including but not limited to, light emitting diode (LED), or liquid crystal display (LCD) monitor or screen, cathode ray tube (CRT) as a visual display; audio signal transducer apparatuses as an audio display; and/or toner, liquid inkjet, solid ink, dye sublimation, inkless (e.g., such as thermal or UV) printing apparatuses, etc.

### Example Embodiments of the Glucose/Insulin Sensor Chip

In some example embodiments, the multi-analyte, multi-detection modality sensor device 100 can be configured as an ImmunoChip strip for decentralized insulin detection in biofluids and a dual-analyte glucose/insulin (GI) sensor chip for detecting simultaneously diabetes markers in a single microliter droplet of a body fluid. As discussed later in this disclosure, the multi-analyte, multi-detection modality sensor device 100 can be fabricated by a specialized masking/sputtering method, which permits the cost-effective and scalable fabrication of chips.

FIG. 1C shows illustrative diagrams and data plots associated with various example embodiment of the sensor device 100 for immunosensing of insulin (e.g., the “ImmunoChip strip”) and multi-modal enzymatic and immunosensing electrochemical detection of glucose and insulin configured in a single sensor chip platform (e.g., the G/I sensor chip). Rapid, decentralized testing of insulin in body fluids is of considerable importance for the management of diabetes; and the sensor device 100 can be implemented to provide such rapid, decentralized diabetes biomarking testing.

In some embodiments, the sensor device 100 includes an immuno-test strip device 140 that includes a detecting electrode 141 (e.g., working electrode (WE)) and a counter/reference electrode 142 (e.g., reference electrode (RE)) on a substrate 149, e.g., for fast and high sensitivity measurement of an analyte in a biofluid, such as insulin in a microliter blood sample. In some embodiments, the detecting electrode 141 and counter/reference electrode 142 includes two thin-film sputtered gold (Au) electrodes disposed on the substrate 149 comprising a plastic material (e.g., PET, PETG, PEN, or PI), where the two gold electrodes are operable as working and counter/reference electrodes, i.e., WE and RE, respectively. The functionalization layer of the immuno-test strip device 140 can be configured in a sandwich electrochemical immunoassay format.

The immuno-test strip device 140 can be operated as an amperometric insulin biosensor in various implementations, which can include in a sandwich electrochemical immunoassay format with (i) horseradish peroxidase (HRP) attached to the detection antibody (D-Ab) used as the enzyme label and (ii) 3,3′,5,5′-tetramethylbenzidine (TMB)/hydrogen peroxide (H₂O₂) as the enzymatic substrate/mediator for the detection system. After formation of a mixed self-assembled monolayer (SAM) 144 on the Au working electrode 141, a capture antibody (C-Ab) 145 specific to the target antigen, i.e., insulin in this example, is covalently attached on the working electrode surface via the SAM 144. The example surface chemistry minimizes non-specific adsorption effects and can help to achieve ultrasensitive insulin detection, e.g., down to the picomolar concentration range. The resulting chip strip can be used for an insulin immunoassay.

In an example implementation of the immuno-test strip device 140, a microliter droplet of the selected biofluid is placed on the insulin immune-strip to cover the two electrodes 141 and 142. This is followed by immuno-reaction with the insulin antigen and the HRP-labeled Ab, to form a sandwich immunocomplex. Successful analysis of insulin detection using the example immune-test strip device 140 has been demonstrated in PBS buffer as well as in complex bodily fluid samples, e.g., such as whole blood or real human saliva without any pretreatment. For example, other surface modification schemes and enzyme labels can be used for such ultrasensitive and selective insulin detection.

In some embodiments, the sensor device 100 includes a dual biomarker sensor chip device 150 that implements two sensing modalities by integration of enzymatic and immunosensing electrochemical detection systems for simultaneous monitoring of at least two biomarkers on the same chip platform. Of particular interest to diabetes management is the simultaneous decentralized measurements of insulin and glucose in the same microliter body fluid, particularly whole blood. Integrating both enzymatic and immunoassay sensing systems with different biosensing operations into a single electrochemical platform required innovation of different bioassay protocols that use the same microliter sample along with different related surface chemistry and bioreceptors. For practical implementations, for example, integration of such bioassays also requires a specific low-cost scalable electrode design to minimize the sample volume needed for bodily fluid analysis. Selectivity toward these two target biomarkers is provided through use of the corresponding chemical and biological modification at the electrode surface (e.g., enzyme or antibody).

The dual biomarker sensor chip device 150 includes a three electrode system comprising a reference/counter electrode 152 (e.g., an Ag/AgCl electrode that acts as both reference and counter electrode), and two detecting electrodes 151, 153. In the example shown in FIG. 1C, the two detecting electrodes 151, 153 include two Au electrodes (e.g., configured as circular electrodes having a 2 mm diameter), which act as dual working electrode transducers for glucose (e.g., WE1 for enzyme biosensor) and insulin (e.g., WE2 for immunosensor), respectively. The localized target analyte detection on the dual biomarker sensor chip device 150 is performed exploiting different surface chemistries for enzyme biosensor WE1 and the immunosensor WE2. The detecting electrodes 151, 153 of the dual biomarker sensor chip device 150 can each include a functionalization layer disposed on or integrated with the electrode to facilitate a reaction and/or conversion of a substance in the biological fluid in a process that is detectable at the detecting electrodes 151, 153. As illustrated in the diagram of FIG. 1C, the dual biomarker sensor chip device 150 includes a catalyst layer 156 on the detecting electrode 151 for enzymatic electrochemical detection of the target analyte; and the dual biomarker sensor chip device 150 includes an immuno-reaction layer that includes a capture antibody 155 (e.g., secured to the detecting electrode 153 via SAM 154).

For example, for the dual biomarker sensor chip device 150 configured as a dual glucose-insulin sensor chip, the catalyst layer 156 on the glucose biosensor WE1 (e.g., detecting electrode 151) includes a glucose oxidase (GOx) layer, which can be fabricated through a layer-by-layer deposition protocol where the enzyme GOx is immobilized inside a permeable polymer film of biopolymer onto the redox mediator. The insulin sensor contingent of the example dual G/I sensor chip can be configured as immuno-test strip device 140, which uses a sandwich immunoassay platform with HRP to be used as the enzyme label and H₂O₂/TMB as the substrate/mediator system. For example, after the formation of the SAM 154 on the gold surface of the detecting electrode 153, the anti-insulin antibody (e.g., C-Ab 155) is covalently attached to the electrode surface (e.g., which can optionally include a subsequent blocking step that is performed to avoid unwanted non-specific adsorptions). The example dual biomarker sensor chip device 150 illustrated in FIG. 1C can be fabricated by the same or similar protocol as the example immuno-test strip device 140 including two Au electrodes 141, 142.

In some embodiments, the sensor device 100 includes a multi-biomarker sensor chip device 160 that implements the two sensing modalities by integration of enzymatic and immunosensing electrochemical detection systems for simultaneous monitoring of a plurality of biomarkers on the same chip platform, e.g., three or more simultaneous biomarker detection. The multi-biomarker sensor chip device 160 includes an electrode system comprising one or more reference/counter electrode 162 (e.g., an Ag/AgCl electrode that acts as both reference and counter electrode), and a plurality of detecting electrodes 161, 163. In the example shown in FIG. 1C, two detecting electrodes 161A, 161B for enzymatic or direct electrochemical sensing are positioned proximate two reference/counter electrodes 162A, 162B, respectively; and two detecting electrodes 163A, 163B for immunosensing are positioned proximate the two reference/counter electrodes 162A, 162B, respectively. In this example, the detecting electrodes 161A, 161B, 163A, 163B include Au electrodes (e.g., configured as circular electrodes). The localized target analyte detection on the multi-biomarker sensor chip device 160 can exploit different surface chemistries for different functionalization layers attached to the electrochemical biosensor and the immunosensor. The detecting electrodes 161A, 161B, 163A, 163B of the multi-biomarker sensor chip device 160 can each include a functionalization layer disposed on or integrated with the electrode to facilitate a reaction and/or conversion of a substance in the biological fluid in a process that is detectable at the detecting electrodes. As illustrated in the diagram of FIG. 1C, the multi-biomarker sensor chip device 160 includes a catalyst layer 166A, 166B on the detecting electrodes 161A, 161B, respectively, for simultaneous enzymatic electrochemical detection of different target analytes. The multi-biomarker sensor chip device 160 includes an immuno-reaction layer that includes capture probes 165A, 165B (e.g., secured to the detecting electrode 163A, 163B, respectively, via SAM 164) for simultaneous immunosensing detection of different target analytes.

FIG. 1D shows a diagram and reaction schemes and data plots illustrating an example implementation of the dual biomarker sensor chip device 150 configured as the example dual G/I sensor chip. As shown in the illustration, once a sample is collected and a 10-μL drop from the sample is placed on the dual G/I sensor chip, the immuno-reaction between insulin analyte, capture antibody and HRP-labeled antibody takes place within a timeframe less than 20 minutes. In some implementations, a washing step is followed by measuring of the amperometric signal. Upon addition of the body fluid (e.g., whole blood), the amperometric measurement of glucose is conducted, e.g., which can be performed 1 minute prior to insulin detection. The amperometric signals registered in both insulin and glucose sensors are proportional to the concentrations of their respective counterparts.

Implementations of the disclosed devices, systems and methods, described herein, demonstrate highly sensitive and selective simultaneous electrochemical detection of glucose and insulin concentrations at physiological ranges of complex bodily fluids. Using the disclosed design principle and fabrication techniques of a fully integrated multi-electrode chip (e.g., for addressing a number of biomarkers to be monitored), the current concept can readily be extended to the simultaneous, continuous and in-vitro multiple biomarker monitoring within bodily fluids including blood, saliva, tears and interstitial fluid (ISF).

Some conventional challenges that are addressed and/or overcome by example embodiments of the sensor chip can include: (1) an inexpensive fabrication method that produces reliable, scalable, and reproducible chips; (2) a chip design that is compact and miniaturized enough to enable detection of small volumes (e.g., <5 uL) in case of body fluid analysis; (3) significant difference between physiological concentration levels of glucose (mM) and insulin (pM); (4) very low pM concentration of insulin in bodily fluids which necessitates a very sensitive immunoassay format; (5) cross-talk between the catalytic and affinity-based sensors especially in terms of both the fabrication protocol and amperometric transduction; (6) different surface chemistries required for each type of sensor; and (7) minimal analysis time, which has to be minimized due to the instability of the biomarkers such as insulin (e.g., laboratories typically require plasma or serum to be separated without delay and stored at −20° C.). Furthermore, minimized detection time is necessary to prevent the potential uncertainties arisen from fluctuation of biomarker levels and also to avoid the coagulation of blood constituents on the electrode surface, which causes interference with the detection signal.

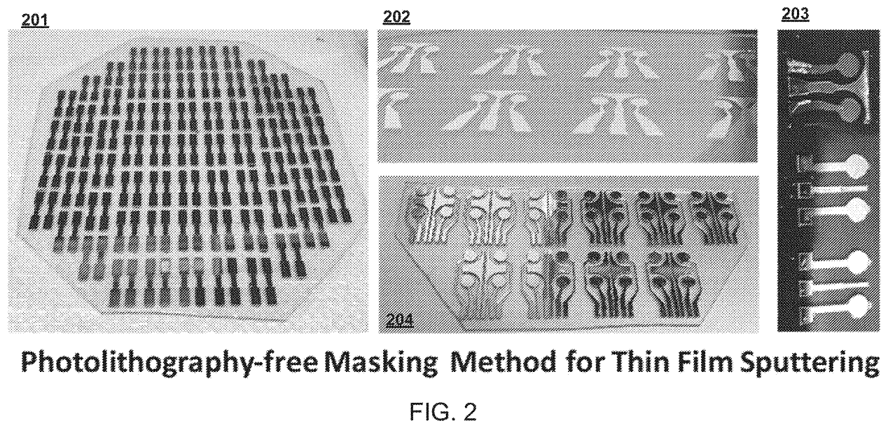

FIG. 2 shows images of different arrays of fabricated sensor chips in accordance with various embodiments of the sensor device 100. Image 201 shows an example array of multiple immuno-test strip devices 140 configured on a single substrate, which can be cut to produce individual immuno-test strip devices 140. Images 202 and 203 show different examples of an array of multiple dual biomarker sensor chip devices 150 configured on a single substrate, which can be cut to produce individual dual biomarker sensor chip devices 150. As shown in images 202 and 203, the physical arrangement of electrodes can be configured in different designs to facilitate a variety of purposes. Image 204 shows an example array of multiple multi-biomarker sensor chip devices 160 configured on a single substrate, which can be cut to produce individual multi-biomarker sensor chip devices 160. The different arrays of sensor chips were fabricated from a photolithography-free masking method for thin film sputtering in accordance with the present technology.

The example embodiments of the G/I sensor chip designs, e.g., the dual biomarker sensor chip device 150 and the multi-biomarker sensor chip device 160, the G/I sensor chip can provide significant value to the personalized insulin monitoring as well as assist in establishing standard levels of typical human insulin levels. This is critically important within the current state of diabetes management technology, as there is currently a lack of reliable reference standards on the blood insulin levels of both diabetic and non-diabetic subjects.

Implementations of the example G/I sensor chips provide a low-cost decentralized sensing method of insulin alone as well as other diabetes markers, e.g., such as glucose or cortisol or ketones. Despite some promising results reported in the literature, all of them failed to meet the aforementioned requirements and suffer significant disadvantages for practical use. For example, previous works mostly performed in-vitro analysis using buffer solution, which can be an indication for the lack of selectivity of those developed sensors that is vital for in-vitro detections using physiological samples such as whole blood, saliva, or ISF. Most existing approaches are directed to sensing these analytes separately. Among the few reports on integration of enzymatic assay and immunoassay toward simultaneous multiple biomarkers detection, the sensitivities are extremely low (e.g., pA and nA current levels for obtained amperometric signals) which makes their real application practically impossible. Also, the extremely complex and time-consuming electrode preparation protocol and instrumentation as well as the analysis time negatively affect both the reproducibility and accuracy of detection signals.

The G/I sensor chip can be fabricated to include smart architecture of different surface chemistries to both avoid any potential cross-talk among the individual sensors (e.g., glucose, insulin and others) and provide a specific and sensitive detection of analytes within the human bodily fluid. In implementations, for example, the sensor chip provides a short detection time, which permits the highly complex insulin measurement task in real samples, using sample volumes below 10 μL.

In some implementations, the sensor chip can detect and monitor other diabetic biomarkers, including but not limited to: β-hydroxybutyrate, which is elevated in diabetic ketoacidosis; cortisol, which is an important stress and diabetes related biomarker; and C-peptide, an 31-amino acid polypeptide, which is employed for differential diagnosis of hypoglycemia or determination of insulin resistance.

In various implementations, the example embodiments of the disclosed sensor chip devices can measure concentrations of multiple analytes simultaneously, upon wetting of the sensor electrodes by the medium containing the targeted analytes. The wide detection range of the sensor chip permits for the medium to be any of the body fluids including whole blood, saliva, ISF and/or sweat. Some implementations described herein demonstrate dual detection of glucose and insulin in Phosphate Buffer Saline (PBS) solution, saliva, and whole blood using a single chip.

Example results of different experimental implementations of the immuno-test strip device 140, the dual biomarker sensor chip device 150 and the multi-biomarker sensor chip device 160 are described below. As demonstrated by the data, the example sensor chip devices have been successfully tested and possess the ability to detect insulin and glucose within human whole blood, and saliva samples. The disclosed electrochemical immunosensor devices of the present technology can be used for the detection of other diabetic biomarkers as well. The integration of an immuno-assay sensor with an enzymatic sensor in such a cost-effective manner using the same sensing platform has opened up new possibilities for detection of multiple analytes in a simultaneous manner using a single chip. The sensor chip of the present technology can be used for diabetic biomarker sensing to more than two analytes.

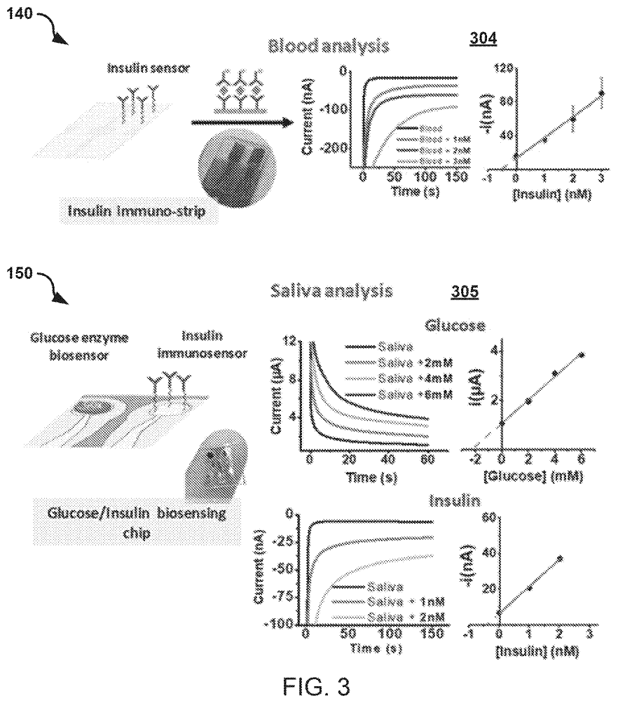

The applicability of the described sensor chips to sensitive detection of biomarkers in body fluids has been successfully demonstrated by analyzing two of the most complex body fluid matrices, e.g., whole blood and raw saliva, where the analysis were carried out in a total time of less than 20 min as shown in FIG. 3. The analytical method is based on the extrapolation of the signal corresponding to the sample on the standard additions calibration plot.

In some embodiments, the sensor chip of the present technology is used for diabetes monitoring, for example, type I diabetes. In some embodiments, the sensor chip can be used in various forms for example, self-monitoring, remote monitoring, and/or use by health providers.

FIG. 3 shows illustrations and data plots depicting example results using example embodiments of the immuno-test strip device 140 for insulin immunosensing and the dual biomarker sensor chip device 150 for simultaneous glucose and insulin sensing. Example implementations of the device 140 included blood analysis, which are shown in the data plots 304 depicting the amperometric response of the example insulin immunosensor to increasing insulin concentrations from 0 to 3 nanomolar (nM) with 1 nM increments. Example implementations of the device 150 included saliva analysis, which are shown in the data plots 305 depicting the amperometric response of the example glucose enzymatic electrochemcal sensing contingent to increasing glucose concentrations in saliva from 0 to 6 nanomolar (nM) with 2 nM increments, and the data plots 305 depicting the amperometric response of the example insulin immunosensing contingent to increasing insulin concentrations in saliva from 0 to 2 nanomolar (nM) with 1 nM increments.

### Example Fabrication Methods of Multi-Analyte, Multi-Modality Sensor Chips

In some aspects in accordance with the present technology, methods for fabricating various embodiments of the sensor device 100 are based on a scalable, on-demand and reproducible non-lithography method, which can be applied to fabricating glucose and insulin biosensors and other multiplex biomarker analysis chips. In some implementations, for example, the disclosed fabrication methods can address and/or overcome the challenges toward the fabrication of enzymatic and immunoassay integrated platform based on oxygen plasma patterning of the chip and can be designed to detect other multiple biomarker detection chips. In some embodiments of the disclosed fabrication methods, a fabricated biosensor chip is integrated with an electrochemical chip with flexible substrates and microneedles for providing real-time continuous monitoring of important multiplex biomarkers.

The examples of the disclosed fabrication methods described below can be used to produce the example glucose and insulin sensor chips, such as the example G/I sensor chip device 150. It is understood the method can be used to produce other multiplex biomarker analysis chips for electrochemical immunosensor devices in accordance with the present technology.

Example Solutions and Reagents. The following provides details regarding the reagents and solutions used in exemplary implementations for the fabrication of a glucose/insulin (GI) sensor according to the present technology. All of the reagents used were of the highest available grade. Glucose oxidase (GOx, from Aspergillus Niger, Type X-S (EC 1.1.3.4)), Chitosan (medium molecular weight), Nafion (perfluorinated resin solution), 11-mercaptoundecanoic acid (MUA), 6-mercapto-1-hexanol (MCH), 1-ethyl-3-(3-(dimethylamino)propyl) carbodiimide (EDC), and N-hydroxysuccinimide (NHS), ethanolamine hydrochloride (≥%99), 2-(N-morpholino)ethanesulfonic acid (YMS), sodium dodecyl sulfate (SDS), tetrathiafulvalene (TTF, %97), iron (III) chloride (FeCl₃), potassium chloride (KCl) and potassium ferricyanide (K₃Fe(CN)₆) were obtained from Sigma-Aldrich. Human Insulin, D-(+)-Glucose and Bovine serum albumin (BSA) were also obtained from Sigma-Aldrich. HPLC grade 2-propanol and Acetone, and ACS grade Hydrochloric acid (HCl) and Nitric acid (HNO₃) were obtained from Fisher Chemical. Pure Ethanol was acquired from KOPTEC. TMB substrate was acquired from Neogen (Enhanced K-Blue TMB Substrate, Neogen Life Sciences, USA). Tween 20 was obtained from Fisher Scientific. Insulin capture antibody and HRP-labeled insulin detection antibody were obtained from US Biological. Phosphate-buffered saline (PBS, 0.1 M, pH 7.4) was prepared by diluting 1.0 M PBS buffer (Sigma-Aldrich) in ultrapure water. Stock solutions of 1 μM Insulin in PBS 0.1 M pH 7.4 containing 32 μM HCl and % 0.02 tween-20, and 1 M Glucose in PBS 0.1 M pH 7.4 were prepared.

Example Equipment and Instruments. The following provides details regarding the equipment and instruments used in exemplary implementations for the fabrication and characterization of an example G/I sensor. Amperometric electrochemical experiments were performed on a μAutolab type III PGSTAT302N (Metrohm), controlled by GPES software v1.11.2, using a two-electrode setup, in which Au and Ag/AgCl electrodes on the chip were employed as working (WE) and reference/counter (RE/CE) electrodes, respectively.

Electrochemical impedance spectroscopy (EIS) measurements were performed on a Gamri Instruments (Interface 1010E) using three electrode setup in which Ag/AgCl (1 M KCl) was used as an external RE, a platinum wire electrode was used as CE, and the Au electrode on the chip was used as WE. EIS investigation was performed in a 0.1 M KCl solution containing 5 mM [Fe(CN)₆]^{3-/4-} at 0.22 V DC potential with 5 mV AC amplitude and the frequencies ranging from 0.1 to 105 Hz.

Example Method of Fabrication of Sensor Chips. The following provides details regarding an exemplary embodiment of the method for fabricating a sensor mask and sensor chips according to the present technology.

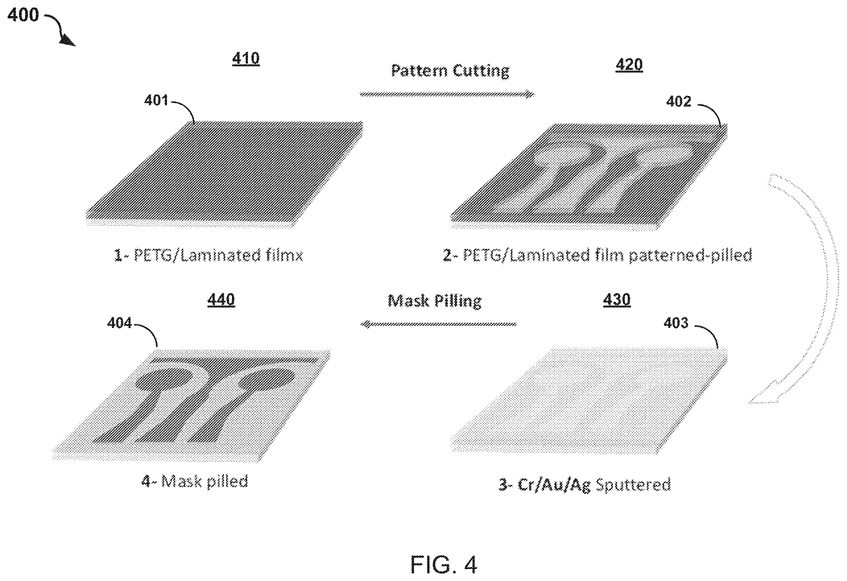

FIG. 4 shows a diagram illustrating an example embodiment of a method 400 for fabricating a multi-biomarker sensor chip in accordance with the present technology. In this embodiment, a sensor chip 404 produced by the method 400 shown in FIG. 4 incudes electrode arrays with two gold (Au) working electrodes (WEs) (e.g., 2 mm in diameter) and a joint silver/silver chloride (Ag/AgCl) reference electrode (RE). The two Au WEs can be used for glucose electrochemical sensing and insulin immunosensing, e.g., as a G/I sensor chip.

In this exemplary embodiment, the method 400 includes a process 410 that provides PETG (polyethylene terephthalate glycol-modified) plastic sheets 401 (e.g., 0.76 mm thickness) to be used as a substrate for electrodes. In some embodiments, the process 410 includes producing a substrate 401 comprising PETG-laminated films. The method 400 includes a process 420 to create a design of a sensor mask 402 by cutting the protective laminated cover of substrates with a Cricut machine to make a pattern for the sensor mask, e.g., PETG-laminated film pattern 402. The method 400 includes a process 430 to deposit an electrically conductive material over the pattern to produce an electrode-material-coated sheet 403 for forming the pattern mask. As illustrated in FIG. 4, in this example, chromium (Cr) and Au metals were sputter-coated, e.g., by using a Denton Discovery 18 Sputter System under direct current (DC) mode at 200 Watts (W) for 20 seconds (s) and 5 minutes (min) for Cr and Au, respectively, under argon (Ar) gas pressure of 2.4 milliTorr (mTorr), followed by radio frequency (RF) sputtering of Ag for 15 min at 100 W and under 2.4 mTorr of Ar pressure to make the Cr/Au/Ag sputtered sheet 403. The method 400 includes a process 440 to produce the sensor chip 404 by removing (e.g., peeling) the sensor mask 404 from the substrate 401 and optionally post-treating the electrode-material patterned on the substrate and/or the substrate material to condition the produced sensor chip 404. In this example, Ag metal layer on each Au WE was etched by reacting with 2 microliters (μL) of a 6 molar (M) HNO₃ solution for 1 min. Each Ag RE/CE was converted to Ag/AgCl through reacting with 2 μL solution of 0.1 M of iron (III) chloride (FeCl₃) for 1 min to make the RE of the sensor chip 404. The electrodes were cleaned before modifications by immersing the electrodes in isopropyl alcohol and water solutions, each for 10 min. Significantly, this process provides a scalable and low-cost method for the fabrication of multi-analyte, multi-modality sensor chips.

Real saliva and whole blood samples collection. In some of the example implementations, saliva samples were obtained from subjects and collected using Sarstedt's Salivettes (Sarstedt, Nümbrecht, Germany). Swabs were chewed for 1 min, after which the swabs were placed into the Salivette's upper cavity and centrifuged immediately for 4 min at 4000 rpm to obtain the available saliva for laboratory use. The whole blood samples obtained from the subject's finger prick without any sample filtration process and pipetted immediately on chip surface for analysis.

Example Techniques for the Glucose Sensor Fabrication and Assay. In some of the example implementations, the glucose sensor was prepared by first forming a tetrathiafulvalene (TTF) mediator layer on Au WE, followed by casting GOx bovine serum albumin (BSA)/Chitosan solution and a sulfonated tetrafluoroethylene polymer, e.g., Nafion. TTF film was formed by drop casting 2 μL of 50 mM TTF prepared in ethanol:acetone (9:1) solution. A %1 Chitosan solution was prepared in %1 Acetic acid solution and overnight magnetic stirred to get a homogeneous solution. GOx (e.g., 20 U/μL) was prepared in a phosphate-buffered saline (PBS) solution containing 10 mg/mL BSA. GOx(BSA)/Chitosan mixture (1:1 V/V) was drop cast on the TTF-modified electrode and left to dry at room temperature. Two layers of 1 μL Nafion solution (%1) were loaded on the electrode surface and left to dry for 4 h at 4° C. The glucose biosensors were evaluated in PBS solution (e.g., 0.1 M, pH 7.4) as well as in whole blood and real saliva samples. Amperometric responses were recorded stepping the potential to +0.2V (vs. Ag/AgCl) for 60 s. Glucose response was measured over mM physiological range of glucose in blood and saliva.

Example Techniques for the Insulin Sensor Fabrication and Assay. In some of the example implementations, the insulin immunosensor was fabricated by immersing the chip electrodes in a mixed solution of 0.1 mM MUA and 1.0 mM MCH in pure Ethanol for 12 h at 4° C. to form a stable thiol self-assembly monolayer (SAM). The electrodes were then rinsed with Ethanol and water and air dried, prior to incubation with 2.5 μL of MES buffer solution (pH 6.5) containing 400 mM (1-ethyl-3-(3-dimethylaminopropyl) carboiimide hydrochloride (EDC) and 100 mM N-hydroxysuccinimide (NHS) for 35 min to activate the carboxylated SAM on the electrode surface. After washing with PBS (e.g., 0.1 M, pH 7.4), the electrodes were incubated with 2.5 μL PBS solution containing 100 μg mL-1 insulin C-Ab for 45 min at room temperature. The electrodes were washed with PBS and dried again, followed by deactivation of the unreacted activated carboxylate groups on the electrode surface by reacting with 2.5 μL of aqueous Ethanolamine solution (e.g., 1 M, pH 8.5). A 2-step sandwich immunoassay format was adopted for insulin measurements through incubation of the C-Ab-modified electrode surface with sample containing Insulin antigen during the first step for 15 min, followed by washing with PBS and 5 min incubation with D-Ab (e.g., 4 μg mL-1) during the second step. The electrodes were washed sequentially with %0.05 sodium dodecyl sulfate (SDS) in PBS and with PBS. The chronoamperometric responses of the electrodes were then recorded in 10 μL 3,3′,5,5′-Tetramethylbenzidine (TMB)/H₂O₂ reagent solution under potential step of −0.1 V for 250 s. The amperometric measurements were performed in a wide concentration range from 0.2 nM to 6.0 nM, which covers almost the whole physiological range of insulin in body fluids.

Example Techniques for the Dual G/I Chip Fabrication Assay. In some of the example implementations, for dual G/I chip fabrication, after Au-etching and immersing the chips in mixed MUA/MCH (1:10) solution, the electrodes were washed in Ethanol and water and dried. The SAM removal on Ag CE/WE was performed by either of three different procedures. In a first approach, Ag electrode was oxidatively treated with 2 μL of NH₄OH:H₂O₂ (2:1) solution for 30 min followed by washing the chip with water. Other tested methods included one based on reductive treatment of CE/WE with a solution of NaBH₄ (0.5M) prepared in ethanol:water (1:1) for 30 min and washing the chip with water; another one based on oxygen plasma exposure involving use of a mask designed to cover and protect the two SAM-modified Au WEs by 3M tape. The unmasked CE/WE was exposed to oxygen plasma in Tepla Asher equipment (PVA TePla America) and treated under 100 W power and 1.2 mbar O₂ pressure for 5 s. After SAM removal on CE/WE, the etching was done by FeCl₃ solution as described above.

The as-prepared electrodes with SAM on Au electrodes are modified stepwise to fabricate G/I chip. For example, first, 2 μL TTF was drop cast on G electrode. This is followed by casting 1 μL GOx(BSA)/Chitosan homogeneous mixture solution and 2.5 μL EDC/NHS on G and I WEs, respectively. After 35 min, the I WE was washed with PBS and dried, followed by putting second 1 μL of GOx(BSA)/Chitosan on G electrode and 2.5 μL C-Ab on I electrode for 45 min. The chips were then modified by 1 μL Nafion on G and 2.5 μL Ethanolamine on I for 30 min. The last step in preparation of G/I chips was casting another 1 μL Nafion on G WE. The fabricated dual analyte chips were stored in fridge until use.

For mixture Glucose/Insulin assays, sample was incubated with G/I mixture solution, for example. The amperometric response was immediately recorded on G sensor at +0.2 V (vs Ag/AgCl) for 60 s. The chips were allowed to incubate for 15 min with the sample solution. Then, chips were washed by PBS and I sensor was incubated with 2.5 μL D-Ab, after which electrode washed with %0.05 SDS in PBS and PBS, and measured in TMB reagent for 250 s at −0.1 V. A control experiment applying the single step sandwich immunoassay format for cross-talk assessment was also performed for insulin measurements through incubation of modified chip with the G/I mixture and spiking 1 μL D-Ab 1 μg mL-1 immediately after G measurement.

Table 1 provides the experimental variables optimized during the fabrication of the G/I sensor in an example embodiment used in the example implementations.

TABLE 1

| | | |
|---|---|---|
| Variable | Evaluated range | Optimum value |

Glucose enzymatic biosensor

| | | |
|---|---|---|
| [TTF], mM | 10-500 | 50 |
| GOx, U | 5-40 | 20 |

Insulin immunosensor

| | | |
|---|---|---|
| [MUA], mM | 0.1-5 | 0.1 |
| [MCH], mM | 0.5-5 | 1 |
| [C-Ab], μg mL⁻¹ | 25-200 | 100 |
| C-Ab incubation time, min | 30-120 | 45 |
| [Ethanolamine], M | 0.5-3 | 1 |
| Number of assay steps | 1-2 | 2 |
| Antigen incubation time, min | 15-90 | 15 |
| [HRP-Ab], μg mL⁻¹ | 1-8 | 4 |

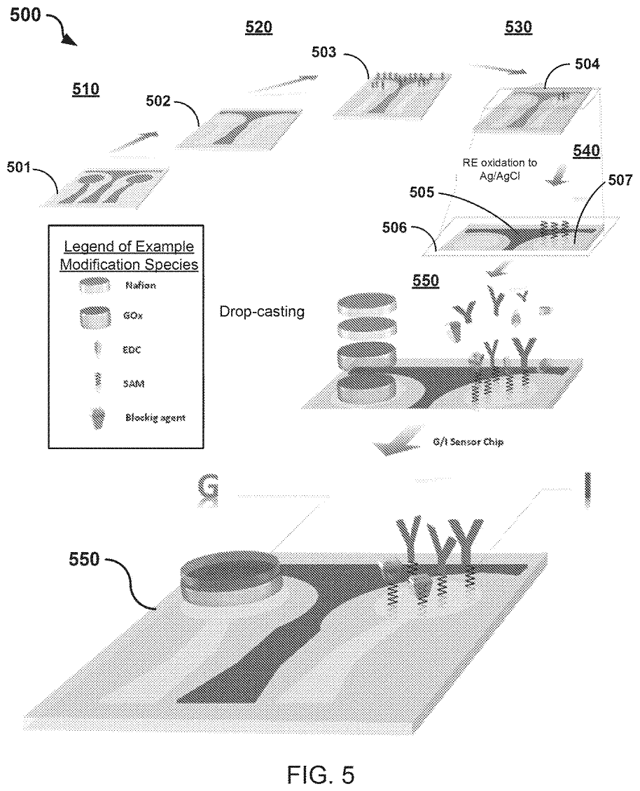

FIG. 5 shows an illustrative diagram depicting an example embodiment of a method 500 for producing a G/I sensor chip 550 in accordance with the present technology. The method 500 includes a process 510 to provide a sensor chip 501 consistent with the exemplary sensor chip 404 produced by the method 400. In some implementations of the process 510, the provided sensor chip 501 can be etched and treated with HNO₃, as needed, to form a treated sensor chip 502 that is ready for electrode surface modifications. The method 500 includes a process 520 to modify the electrode material of the treated sensor chip 502. In some embodiments, the process 520 includes exposing the treated sensor chip 502 to a solution containing SAMs such that SAMs form on metals electrodes (e.g., Au) resulting in a SAM-modified sensor chip 503. The method 500 includes a process 530 to selectively remove some of the SAMs on certain electrode structures of the SAM-modified sensor chip 503, e.g., resulting in the formation of the selective-SAM-modified sensor chip 504. As illustrated in FIG. 5, the selective-SAM-modified sensor chip 504 includes a SAM-modified electrode 507 on one of the detecting electrodes. The method 500 includes a process 540 to modify the reference electrode (RE) of the selective-SAM-modified sensor chip 504. For example, the RE of the selective-SAM-modified sensor chip 504 is oxidized to Ag/AgCl to form an oxidized layer 505. The method 500 includes a process 550 to functionalize the electrodes of the oxidized/SAM-selectively modified sensor chip to produce the G/I sensor chip 550. In some implementations of the process 550, the oxidized/SAM-selectively modified sensor chip 506 is functionalized via vast drop casting, and the SAMs are then modified, resulting in the G/I sensor chip 550. The drop-casing modification technique of the example embodiment of process 550 can include drop-casting of a first modification layer on a first electrode for electrochemical sensing (e.g., “G electrode”); where, in some examples, TTF is drop-casted on the G electrode. The drop-casting modification technique can then include drop-casting a second modification layer on the first electrode; where, in some examples, a enzymatic-sensing layer of comprising GOx(BSA)/Chitosan is drop-casted on the G electrode. In some implementations, in parallel or sequentially, a modification layer on the SAM-modified second electrode (e.g., “I electrode”) is produced; where, in some examples, the C-Ab is attached to the I electrode. In some implementations, the sensor chip is then modified by Nafion on the G electrode and Ethanolamine on the I electrode, e.g., via drop-casting. In some implementations, Nafion drop-casting can be repeated to prepare another layer.

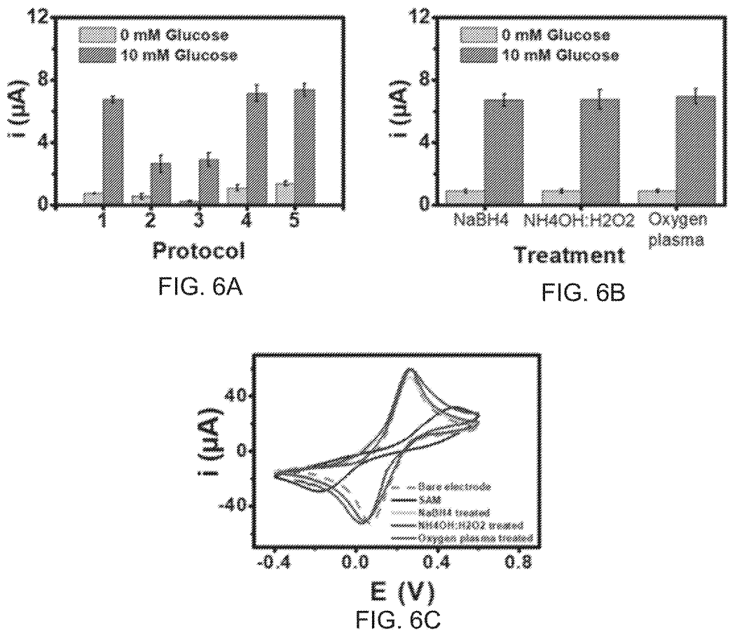

FIGS. 6A-6C shows data plots regarding the evaluation of the performance of the example G/I sensor chip fabricated by the method 500. FIG. 6A shows the amperometric responses obtained with the example G biosensor electrode by chronoamperometry in PBS 0.1M, pH 7.4 at +0.2V for 0 and 10 mM glucose using different chip fabrication protocols, e.g., (1) Control chip without SAM modification; (2) SAM on both G WE and CE/RE; (3) G WE treated with NH₄OH:H₂O₂ before Ag etching, SAM on AgCl CE/RE; (4) SAM on G WE, Ag CE/RE treated with NH₄OH:H₂O₂ before chloridation with FeCl₃ to AgCl; (5) Both G WE and CE/RE treated with NH₄OH:H₂O₂ before Ag etching and chloridation, respectively. FIG. 6B shows the amperometric responses obtained with the example G biosensor electrode by chronoamperometry in PBS 0.1M, pH 7.4 at +0.2V for 0 and 10 mM glucose concentrations using different treatments for removing SAM from G-WE and RE before etching and chloridation with FeCl₃ to AgCl, respectively. In FIGS. 6A and 6B, the column bars data and error bars represent the average and standard deviations from three independently fabricated electrodes. FIG. 6C shows a data plot depicting the recovery of the electrode surface by treatment of SAM modified Au, depicting Cyclic voltammograms (CV) of bare Au electrode (dashed line plot), SAM modified Au (black line plot), NaBH₄ treated electrode (green line plot), NH₄OH—H₂O₂ treated electrode (purpl line plot e) and oxygen plasma treated electrode (blue line plot). CVs were obtained in 0.1 M KCl solution containing 5 mM Fe(CN)₆^{3-/4-}. In FIG. 6C, the frequency range was 0.1-105 Hz with Amplitude of 5 mV and constant potential of 0.22 V.

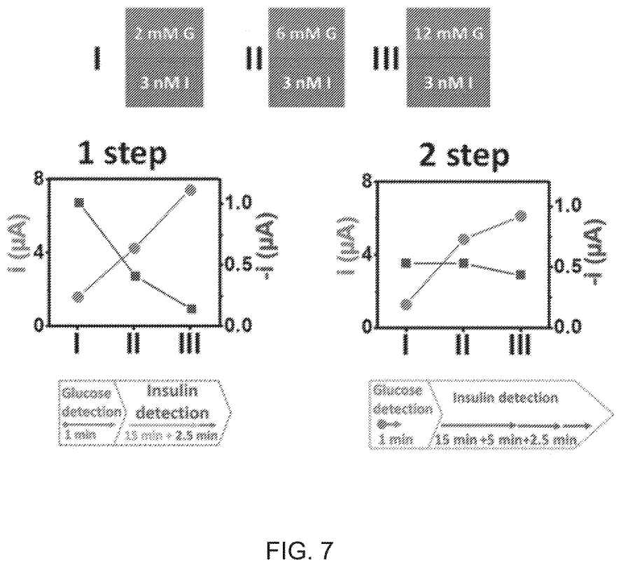

FIG. 7 shows plots depicting the amperometric responses measured with G/I chips for the comparison of one-step vs. two-step incubation protocols regarding I immunossay to confirm cross-talk between G and I sensors. In the example one-step protocol: D-Ab is introduced immediately after glucose measurement and incubated along with insulin in the sample during 15 min. In the example two-step protocol: immediately after glucose measurement, the sample is incubated for 15 min for insulin antigen recognition by the C-Ab and then in a separate step the D-Ab is incubated for 5 min. For each example protocol, G/I signals were recorded for different chips when exposed to constant insulin concentration of 3 mM, and varying glucose concentrations 2, 6, and 12 mM.

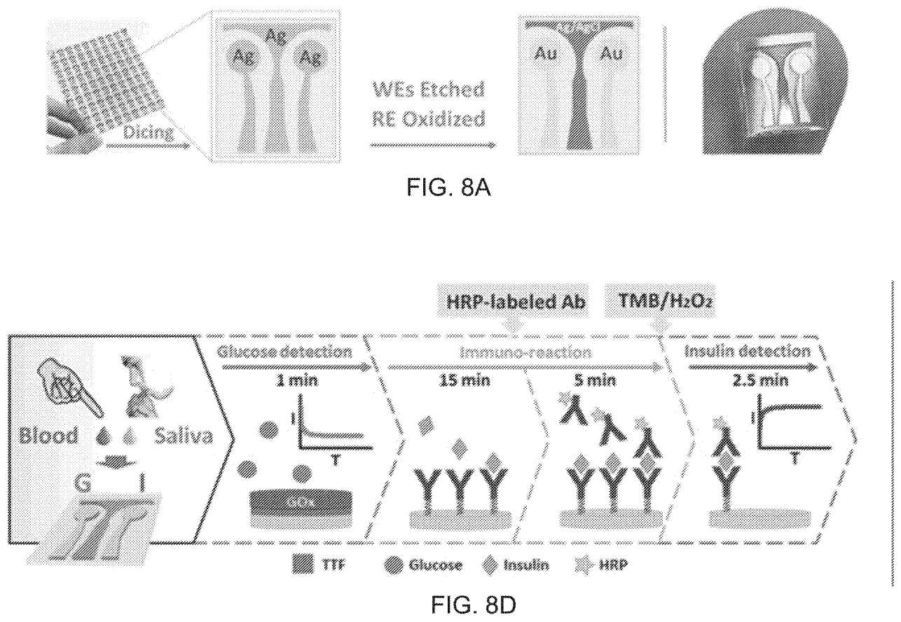

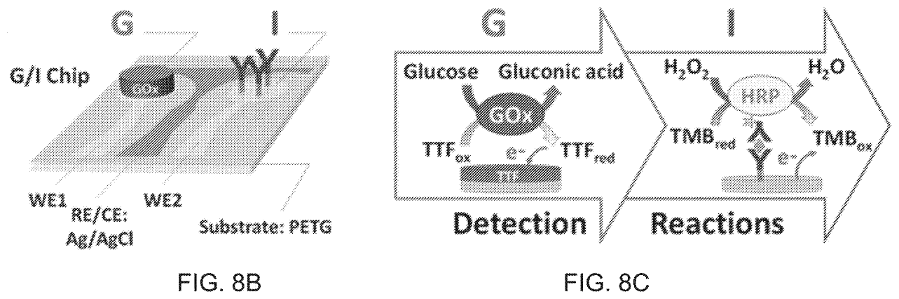

In some implementations, the example dual G/I sensor chip 150 was designed in a three-electrode system, comprising of thin-film sputtered silver and gold electrodes on a plastic PETG substrate as shown in FIGS. 8A-8D. In the exemplary embodiment of the G/I sensor as shown in FIGS. 8A-8D, the sensor chip includes a joint silver/silver chloride (Ag/AgCl) reference/counter electrode (RE) and two gold (Au) working electrodes (WEs) as transducers for glucose and insulin detection.

FIG. 8A shows a diagram illustrating the production of an array of fabricated glucose insulin sensor chips on a PETG substrate, which can be produced in accordance with the method 400. In FIG. 8A, the diagram shows each chip of the array includes two Au working electrodes that can be modified for the glucose biosensor electrode and the insulin immunosensor electrode, with an Ag/AgCl electrode acting as a joint RE/CE.

Modification of the biosensor electrode and immunosensor electrode for localized detection of the insulin and glucose targets is shown FIGS. 8B and 8C, where different recognition and surface chemistries are exploited for each analyte (e.g., insulin and glucose). As shown in the diagram of FIGS. 8B and 8C, the glucose biosensor is fabricated through a layer-by-layer deposition protocol as shown in where the glucose oxidase (GOx) enzyme is immobilized within a permeable biopolymer chitosan film and onto the TTF redox mediator layer on WE1. Also, in this example, the insulin sensor relies on a sandwich immunoassay format using horseradish peroxidase (HRP) as enzymatic label and TMB/H₂O₂ as the mediator/substrate detection system. The insulin capture antibody (C-Ab) is covalently attached to carboxylic groups of the mixed self-assembled monolayer (SAM) on WE2 via EDC/NHS coupling, along with surface blocking with ethanolamine. This is followed by two-step incubation of insulin and HRP-labeled detection antibody (D-Ab) for 15 and 5 min, respectively, to form a sandwich immunocomplex of insulin and to capture the D-Ab.

FIG. 8C illustrates the mechanisms involved in each sensing platform. The GOx-based biocatalytic detection of glucose relies on the electron-carrying mediator TTF, and the reoxidation of the reduced form of TTF to yield current response proportional to the glucose concentration. In the case of insulin immunosensor, the Ab-modified electrode is incubated in different insulin concentrations, followed by incubation with the D-Ab, and introduction of the TMB/H₂O₂ detection solution. HRP thus catalyzes the reduction of H₂O₂ coupled to the oxidation of TMB into its oxidized form. The corresponding H₂O₂ reduction signal is thus directly proportional to the amount of the captured HRP tag and hence to the insulin concentration.

The resulting electrochemical chip is operable for the simultaneous dual-marker G/I detection in different body fluids samples, e.g., within less than 30 min, as illustrated in FIG. 8D. In this example, the G/I sensor chip detects each biomarker in blood and saliva fluids as demonstrated using complex untreated whole blood and human saliva sample. A single sample droplet (e.g., 10 μL) was cast over the sensor chip to cover the entire electrode area. Glucose is thus analyzed amperometrically on WE1 within the initial 60 seconds, while the insulin recognition requires additional 15 min incubation for binding with the anti-insulin antibody. Subsequently, to complete the sandwich immunoreaction, a 5 min incubation with D-Ab is performed, followed by amperometric detection of the H₂O₂/TMB redox probe.

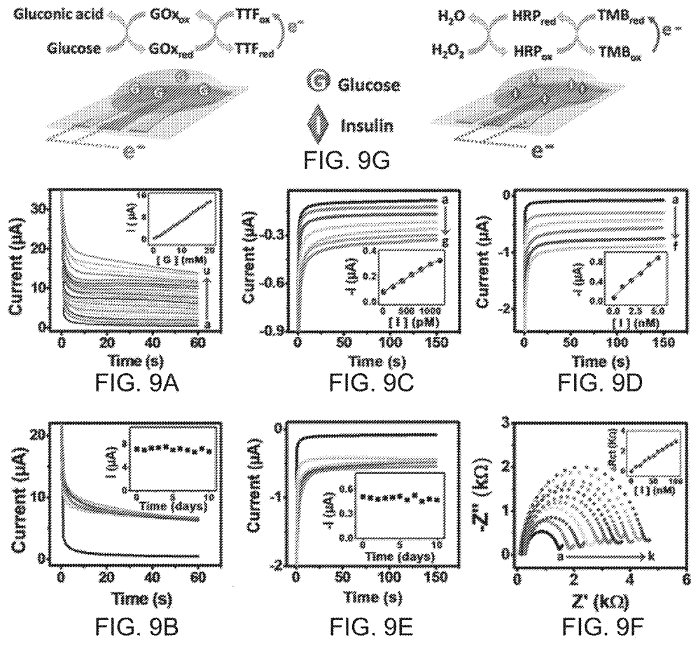

Example experiments to evaluate the performance of the example G/I sensor ship were conducted, with example results depicted in FIGS. 9A-9G. These results are discussed for this particular example.

FIGS. 9A-9F show data plots depicting the sensing performance of an example G/I sensor chip, corresponding to the illustration of the G/I sensor chip shown in FIG. 9G. Specifically, FIG. 9A depicts the amperometric response of the enzymatic G sensor to increasing concentrations of glucose from 1 (a) to 20 mM (u) with 1 mM increments. FIG. 9B shows the reproducibility of the G sensor to 10 mM glucose detection with the inset showing the 10 mM glucose signal glucose signal monitoring during 10 days from fabrication. FIG. 9C depicts the amperometric response of the I immunosensor to increasing insulin concentrations from 0 (a) to 1200 pM (g) with 200 pM increments. FIG. 9D shows the amperometric response of the I immunosensor to increasing insulin concentrations from 0 (a) to 5 nanomolar (nM) (f) with 1 nM increments. FIG. 9E shows the reproducibility of the I immunoassay for 3 nM insulin with the inset showing the stability of I immunosensor, corresponding to signals for 3 nM insulin during 10 days from fabrication. FIG. 9F are Nyquist curves of the I immunosensor upon increasing the I concentration over the range 0-100 nM (a-k) with 10 nM increments.

The electrochemical sensing performance and operational characteristics of the dual biosensor chip were evaluated as shown in FIGS. 9A-9F after optimizing the experimental conditions for each sensor as detailed above in Table 1. The analytical performance of the G/I sensor chip was assessed first by constructing calibration plots for both analytes in PBS. The enzymatic G sensor was characterized first from its amperometric response for millimolar glucose solutions as shown in FIG. 9A, whereas the insulin immunosensor was evaluated both in the pM and nM concentration ranges as shown in FIGS. 9C and 9D, respectively. These data yield limits of detection (LODs) of 0.2 mM glucose and 41 pM insulin. The analytical performance of the insulin sensor was examined also by label-free EIS immunosensing approach using Nyquist curves for increasing insulin concentrations between 10 and 100 nM as shown in FIG. 9F, which results in a LOD of 8.9 nM insulin. Such label-free immunosensing approach results in approximately 200-fold lower sensitivity compared to the HRP-labeled sandwich based amperometric immunosensor. The latter thus meets the demands of highly sensitive pM insulin detection in body fluids.

The reproducibility of the amperometric sensor response was assessed by testing five different glucose and insulin biosensors constructed using the same fabrication protocol. Chronoamperometric measurements recorded for 10 mM glucose in FIG. 9B and 3 nM insulin in FIG. 9E yielded RSD values of 2.0% and 8.9% respectively, confirming the reliability of the chip fabrication and operation. Additionally, the storage stability of the G and I sensors was evaluated by monitoring the amperometric response for 10 mM glucose and 3 nM insulin. Both sensors were thus stored at 4° C. and monitored during 10 days. The highly stable glucose and insulin response observed over these 10-day periods as shown in FIGS. 9B and 9E, respectively demonstrate that such storage has no apparent effect on the sensitivity.

The example dual G/I sensor chip, illustrated in FIG. 9G, can be fabricated through optimal control of the modification protocols (e.g., as shown and discussed in connection with the method 500 in FIG. 5). The primary fabrication challenge addressed is the fundamentally different surface (receptor-immobilization) chemistries required for the adjacent G and I sensors on the same single chip. As shown in FIG. 9A, initial results showed that the presence of SAM, necessary for the I immunosensor, suppresses the response of the adjacent G sensor. Further experiments showed that SAM on RE should be removed in order to preserve an optimal current response. This may be ascribed to an increasingly sluggish oxygen reduction as the cathodic half-reaction on RE surface in the presence of SAM. In contrast, TTF and GOx enzyme can be co-immobilized on the SAM-modified G WE without compromising the enzymatic activity and detection, with the glucose signal independent of the presence of SAM on WE surface. During the fabrication development procedures, various methods were examined for removing the SAM from the metallic surface of the sensor as shown in FIGS. 9B and 9C. These included oxygen plasma treatment, chemical reduction with NaBH₄ and chemical oxidation by using NH₄OH:H₂O₂. All treatments were suitable for removing SAM; however, because of the nature of oxygen plasma and NaBH₄ treatment methods which require sealing I WE by masking, the most practical and safe SAM removal protocol was found to be oxidizing the SAM with NH₄OH:H₂O₂. It is also worth noting that Ag RE conversion into Ag/AgCl is performed after removing the SAM as the opposite order led to destruction of the conductive nature of electrode, which may be attributed to an irreversible partial transition of AgCl to Ag₂O. Another challenge relates to spreading of acetone/ethanolic solution of TTF during immobilization by drop casting to neighboring electrodes. This was addressed through optimizing the spatial separation of the electrodes to 2 mm to avoid spreading of TTF to the I sensing electrode.

In order to demonstrate the applicability of the fabricated G/I sensor chips toward assays of microliter sample mixtures, example implementations were performed that included testing different G-I mixtures of varied concentrations of G and I, e.g., in the ranges of 2-18 mM and 0.5-3 nM, respectively. For this purpose, an example two-step incubation protocol was adopted for the insulin immunoassay.

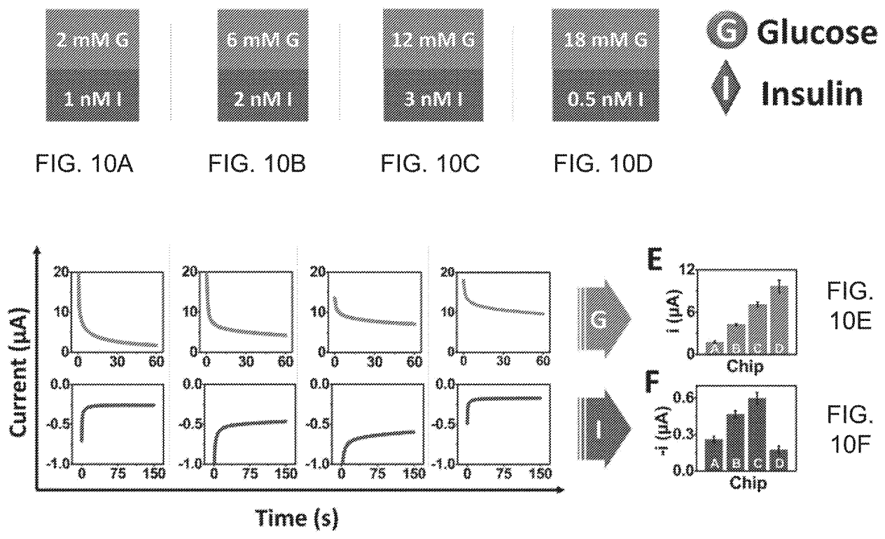

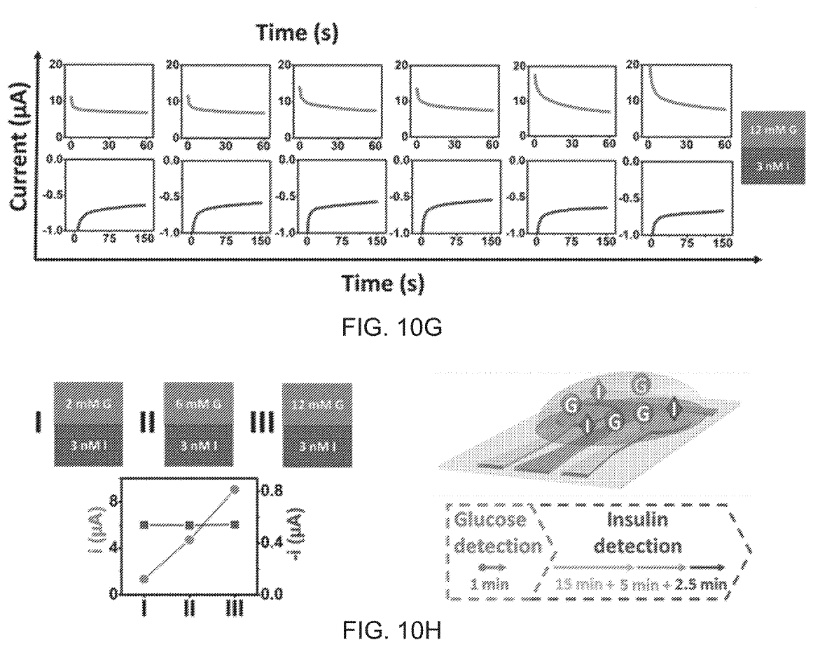

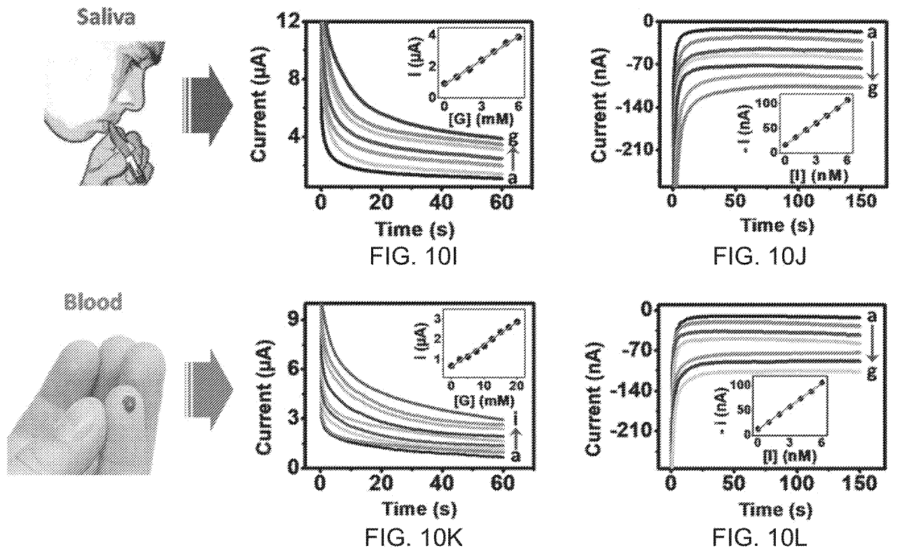

FIGS. 10A-10D display the signals obtained for glucose (red curves) and insulin (blue curves) on four different chips exposed to these different G/I mixtures of varying concentrations. FIG. 10A corresponds to 2 mM G/I nM I. FIG. 10B corresponds to 6 mM G/2 nM I. FIG. 10C corresponds to 12 mM G/3 nM I. FIG. 10D corresponds to 18 mM G/0.5 nM I.

Glucose amperometric measurements were carried out during the first minute, while insulin was detected after 15 min incubation with the same sample followed by 5 min incubation with HRP-labeled D-Ab. Well-defined amperometric signals were obtained for all cases, indicating minimal cross-talk and successful G/I measurements using single dual-analyte chips as shown in FIGS. 10E and 10F.

Such protocol offers also high reproducibility, as demonstrated in FIG. 10G for six different chips with a 12 mM glucose/3 nM insulin sample mixture (RSD values of 5.0% and 8.3% for G and I sensors, respectively.

The two-step vs. one-step immunoassay incubation protocols towards G/I mixture analysis were compared; the results of which are shown in the results of which are shown in FIG. 10H and FIG. 7. In the one-step protocol, the D-Ab is introduced immediately after glucose measurement and incubated along with insulin in the sample during 15 min. For each protocol, the G/I signals were recorded for different chips when exposed to a fixed (3 nM) insulin concentration and increasing glucose levels (e.g., 2, 6, and 12 mM). The two-step protocol resulted in similar insulin response along with increasing glucose signals. For example, the one-step method resulted in decreased current values for insulin upon increasing the glucose concentrations. While H₂O₂ is not directly involved in our mediated glucose detection mechanism, it can still be produced in the presence of GOx enzyme with glucose and oxygen in the solution, and affect the activity of the captured HRP enzyme tag of D-Ab during the 15 min incubation step, ultimately leading to a false-negative insulin response. This mechanism was further confirmed by a two-step protocol in which D-Ab was incubated separately on the chip. In this case, for example, increasing G level in the mixture did not impact the insulin signal as shown in FIG. 10H.

The ability to detect multiple biomarkers in as-received body fluids, without any sample treatment can have a profound impact on decentralized clinical screening. The example G and I sensors were tested individually using untreated whole blood and raw saliva samples. The saliva collected by a Salivette® and the finger-stick capillary blood sample were directly pipetted on the electrode surface for analysis. The saliva sample was spiked with increasing 1 mM glucose and 1 nM insulin concentrations, displayed a well-defined response in FIGS. 10I and 10J, respectively, with of LOD 209 μM G and 340 pM I.

FIGS. 10I-10L show data plots evaluating the performance of example electrochemical sensing of an example embodiment of the glucose/insulin sensor chip using blood samples (FIGS. 10K, 10L) and saliva samples (FIGS. 10I, 10J). FIG. 10I shows the amperometric response of the G sensor to increasing concentrations of glucose from 0 (a) to 6 mM (g) with 1 mM increments in real human saliva; and FIG. 10J shows the amperometric response of I immunosensor to increasing concentrations of insulin from 0 (a) to 6 nM (g) with 1 nM increments in real human saliva. Similarly, using the capillary blood, the example multi-modal analyte detection G/I sensor chip offers convenient measurements of the increasing 2.5 mM glucose and 1.0 nM insulin concentrations, as shown in the data plots of FIGS. 10K and 10L, with LOD values of 674 μM G and 300 pM I. Despite the lower sensitivity observed with blood and saliva samples, e.g., as compared to PBS (owing to matrix effects), the example G/I sensors demonstrated a very attractive biosensing performance, leading to a first demonstration of strips capable of G/I measurements in body fluids. FIG. 10K shows the amperometric response of G sensor to increasing concentrations of glucose from 0 (a) to 20 mM (i) with 2.5 mM increments in whole blood; and FIG. 10L shows the amperometric response of the I immunosensor for increasing insulin concentrations from 0 (a) to 6 nM (g) with 1 nM increments in whole blood.

The above example embodiments and implementations of a G/I sensor chip device illustrate the capability of a single electronically-addressable sensor chip incorporating fundamentally different enzymatic and immunoassays of microliter sample mixtures and demonstrate the analytical attractive performance toward simultaneous point-of-care detection of key diabetes biomarkers (e.g., insulin and glucose). Such single-chip G/I sensing platform has been realized by judiciously addressing key operational and fabrication challenges associated with the different surface chemistries and procedures of these two different bioassays, and the significantly different (e.g., >million fold) G and I concentration levels. The sensor chip of the present technology can be fabricated with low-cost non-lithography techniques and improve decentralized diabetes assays. Ultimately, such coupling of enzymatic and immunoassays of metabolites and protein biomarkers onto single microchip sensing devices opens up new possibilities for point-of-care screening and diagnosis of various diseases and disorders and for variety of environmental and biodefense applications.

### Example Embodiments of a Simultaneous Cortisol/Insulin Microchip-Based Sensor Using Dual Enzyme Tagging

In some embodiments of the multi-analyte, multi-detection modality sensor device 100, a dual biomarker sensor chip device 1150 is configured to simultaneously detect cortisol and insulin by two sensing modalities via integration of enzymatic and immunosensing electrochemical detection systems on the same chip platform. Of particular interest to diabetes management is the simultaneous decentralized measurements of insulin and cortisol in the same microliter body fluid, e.g., a blood serum sample. Described below are example embodiments and implementations of the dual biomarker sensor chip device 1150 for cortisol and insulin sensing, e.g., also referred to as a cortisol/insulin sensor chip or C/I sensor chip.

The dual biomarker sensor chip device 1150 can include any of the example designs of the sensor chip of the dual biomarker sensor chip 150 described above. In some example embodiments of the dual biomarker electrochemical sensor chip 1150, the sensor chip 1150 is configured with a specialized functionalization layer for simultaneous detection of cortisol and insulin that combines competitive and sandwich immunoassay formats and fundamentally distinct enzymatic labeling strategies based on ALP and HRP for amperometric readout. Example implementations of the simultaneous electrochemical cortisol/insulin-detecting sensor chip 1150 are described with exemplary data demonstrating its analytical performance of the dual biosensing platform, which included the judicious optimization of the system for detection of these biomarkers in a minimum sample volume that are at considerably different concentration in body fluids and the potential cross-talk related to the electrochemical transduction and biorecognition conditions. The example dual biomarker sensor platform demonstrated a very attractive performance in the simultaneous detection of cortisol and insulin in a microliter sample drop in an assay shorter than 25 min towards sensitive detection of these diabetes biomarkers in body fluids.

Diabetes, a heterogeneous disorder of glucose metabolism, is a major public health problem with epidemic proportions affecting millions of people around the world. In 2014, the World Health Organization reported that there was approximately a worldwide number of 422 million adults living with this condition. It has been estimated that about 18 million people die every year from cardiovascular disease, for which diabetes and hypertension are major predisposing factor. Diabetes positively has become one of the most challenging health problems in the 21st century being the seventh leading cause of death in the United States. Complications from this disease such as coronary artery and peripheral vascular disease, stroke, diabetic neuropathy, lower-extremity amputation, renal failure and blindness lead to increasing disability, reduced life expectancy and enormous health costs for every society. Considering the prevalence and impact of diabetes, large international organizations are dedicated to the treatment and, ultimately, the cure of diabetes and its complications, including: ADA (American Diabetes Association), the European Association for the Study of Diabetes (EASD), the International Diabetes Federation (IDF), and the Diabetes Technology Society. In this context, major efforts focus on diabetes care and management development have been made by scientific community over the last decade towards better understanding, earlier recognition, effective personalized glucose monitoring and more aggressive treatment of diabetes and diabetic risk factors and complications. The existence of a cluster of metabolic disorders in people with problems to metabolize glucose triggered by nutritional excess and reduced levels of activity in the setting of genetic predisposition have been widely reported. In addition, the physical characteristics and lifestyle of the individual must be considered as essential conditioning factors for effective diabetes control and personalized treatment.

Among the clinical parameters studied in the characterization of diabetes, such as glucose, lactate, ketone bodies, insulin and C-peptide, glucocorticoids are known to be among the various conditions and factors involved in the pathophysiology of diabetes through an excess of glucocorticoids, from their administration or in pathological conditions such as Cushing syndrome, leading to diabetes. These steroid hormones promote gluconeogenesis by inducing the expression of gluconeogenic genes in the liver so that, by suppressing glucose uptake in skeletal muscle and adipocytes, insulin resistance is induced. Therefore, insulin resistance has been considered as a probable mechanism by which an excess of glucocorticoids leads to diabetes. Similarly, although less known, whether an excess of glucocorticoids affects pancreatic β-cell function and insulin secretion is being studied, since insulin secretion increases compensatory along with increases in insulin resistance to maintain plasma glucose levels as normal as possible.

Cortisol plays an important role in the regulation of various physiological processes such as blood pressure, glucose levels, and carbohydrate metabolism. It has been investigated as a biomarker for numerous diseases since increased circulating levels of cortisol has been related to metabolic disturbances such as hypertension, hyperlipidemia, and central obesity, and may lead to worsening of insulin resistance, glucose intolerance, overt diabetes mellitus and cardiovascular disease. The identification of mechanisms that determine the regulation and specifically dysregulation of free cortisol responses to stress since stress and stress-related health impairments linking other medical conditions such as diabetes is also of major importance, so that effects of stress on blood pressure and glucose homeostasis may be enhanced as a result of lipid-induced insulin dysfunction for instance. Thus, with the aim of transforming clinical practice and improving the understanding of its biological effects, the role of cortisol on the insulin mechanisms and pathways and glucose metabolism has been extensively explored towards circadian modulation of glucose and insulin responses to meals and still needs to be elucidated.

Fortunately, efforts aimed at optimizing diabetes control are accelerating and new diabetes treatment technologies are being introduced, such as the development of artificial pancreas and closed loop systems that automatically sense blood glucose and precisely adjust insulin dosage. However, there is still an acute need for the development of sensor devices that allow on-the-spot multiplexed monitoring of several biomarkers in diabetes control avoiding the delay between sampling and analysis and data acquisition. In the case of insulin and cortisol, two biomarkers of great relevance in the characterization of diabetes, a significant drawback of the current set-up is that they only provide a snapshot of the insulin and cortisol levels of the samples sent to a diagnostic laboratory and do not provide a true representation of the variations of insulin and cortisol that a specimen undergoes in a certain environment that triggers the generation or suppression of these biomarkers. Therefore, in situ monitoring of insulin and cortisol levels is required to obtain valuable information that could help physicians to better diagnose and treat diabetes-related conditions. Most current strategies for the analysis of these biomarkers are limited to laboratory techniques that are laborious, time-consuming, require large sample volume, expensive, and cannot be implemented at point-of-care. Thus, there is a need to develop sensing platforms for on-the-spot detection of cortisol and insulin, whose characteristics enable its application as point of care device and thus avoiding clinics to outsource analysis. In other words, it should be portable, disposable, sterile, miniaturized, require minimal sample volume, low energy consumption, have a low response time and be cost effective.

As discussed below, embodiments of the dual biomarker sensor chip device 150 and/or the multi-biomarker sensor chip 160 provide a novel electrochemical sensor chip for the simultaneous detection of two or more diabetes biomarkers. In particular, embodiments of the example C/I sensor chip device 1150 for detecting cortisol and insulin, two key diabetes biomarkers, are described with respect to example implementations, e.g., demonstrating how the example C/I sensor chip device 1150 can detect cortisol and insulin in less than 25 min in a microliter sample drop. Moreover, embodiments of an example fabrication method for producing the example C/I sensor chip device 1150 is described, e.g., demonstrating a new straightforward, scalable, and low-cost non-lithography-based technology.

The simultaneous dual cortisol/insulin analyte immunoassay developed is based on a dual competitive and sandwich format assay, respectively, and the use of a multiple labeling strategy by the ALP and HRP enzymes to convert the biorecognition events into the electrode surface in sensitive electrochemical signals. In order to effectively measure cortisol and insulin in practical applications, meticulous and detailed optimization of the system was carried out so that the integration of both electrochemical biosensing approaches was achieved by exploring and addressing the challenges regarding the detection of two biomarkers in a minimum sample volume that are at considerably different levels in body fluids (pM for insulin and nM, which corresponds to ng mL⁻¹ range, for cortisol) and the potential cross-talk related to the electrochemical transduction and biorecognition conditions. As shown by the example data presented below, the example C/I sensor chip device 1150 successfully performed in simultaneous detection of this biomarkers in untreated serum samples.

### Example Fabrication Methods of C/I Sensor Chips

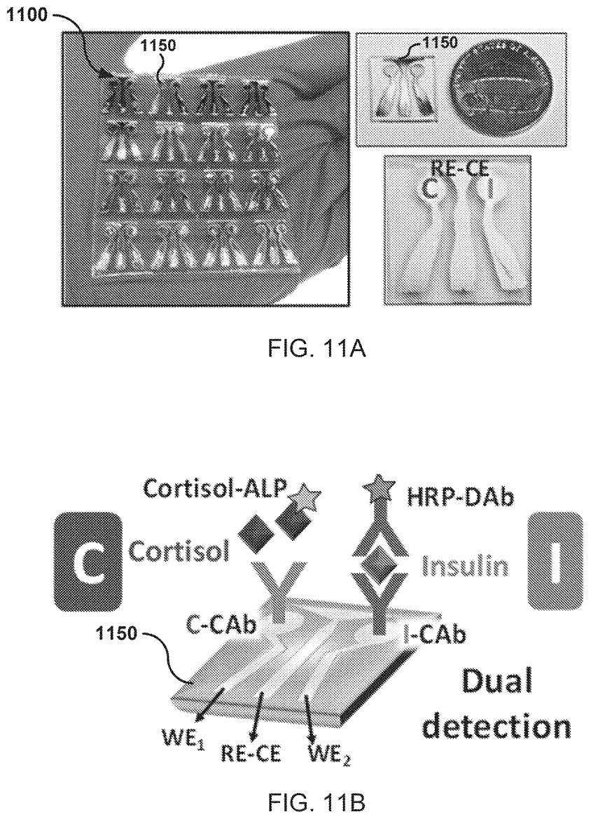

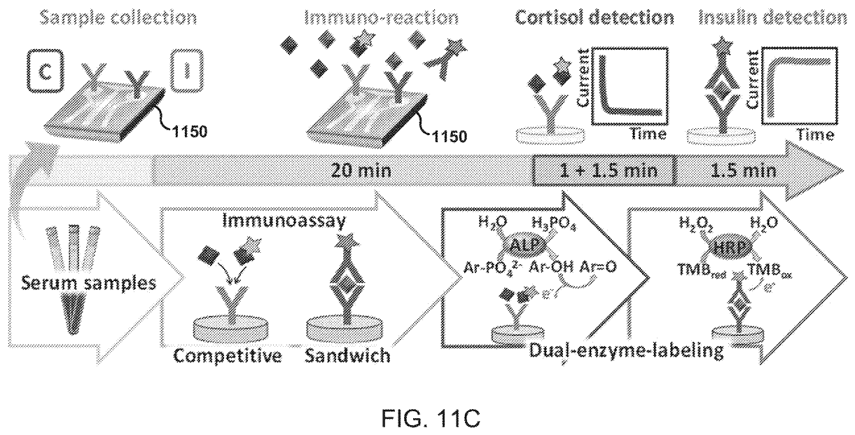

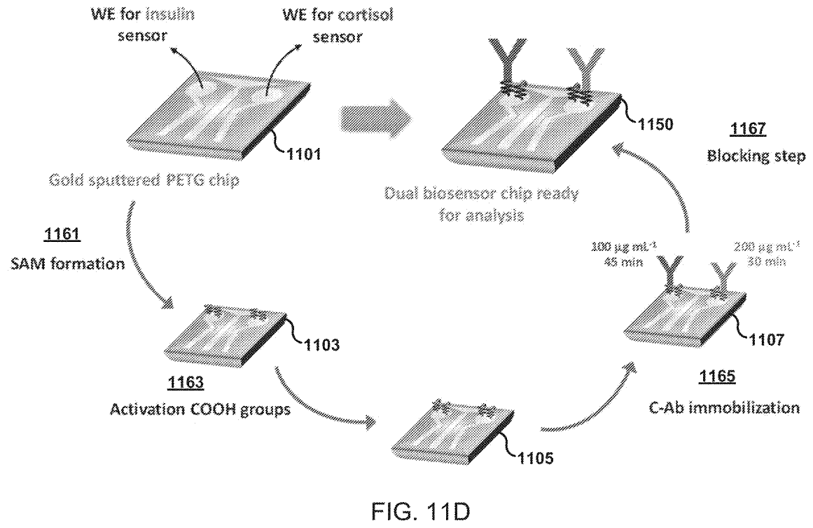

Example embodiments of the C/I sensor chip device 1150 and method for fabricating the example C/I sensor chip device 1150 are shown in FIGS. 11A-11D and described below. FIG. 11D shows an example embodiment of a method 1160 for fabricating the C/I sensor chip device 1150, and an example implementation of the method 1160 is described in the paragraphs below.

Example Equipment and Instrumentation. For example, a Barnstead Thermolyne (Type 16700 Mixer) vortex for homogeneization of the solutions and pH/ISE meter (Orion Star A214 model) from Thermo-Scientific were used. Chronoamperometric responses were obtained using a μAutolab type III PGSTAT302N potentiostat (Metrohm) controlled by GPES software v1.11.2. Example experiments were performed with homemade dual chip electrodes, which included two working electrodes (e.g., 2.0 mm diameter each) and a central joint counter-reference electrode, in both cases made by sputtered gold.

Example Reagents, Solutions and Samples. The example reagents used in this work were of the highest purity grade. Commercial phosphate buffer solution (1.0 M), 11-mercaptoundecanoic acid (MUA), 6-mercapto-1-hexanol (MCH), 1-ethyl-3-(3-(dimethylamino)propyl) carbodiimide (EDC), N-hydroxysuccinimide (NHS), ethanolamine hydrochloride (≥%99), 2-(N-morpholino) ethanesulfonic acid (MES), 1-Naphthyl phosphate (1-NPP) and sodium dodecyl sulfate (SDS) were acquired in Sigma-Aldrich. HPLC grade 2-propanol and sodium borate were obtained from Fisher Chemical and pure ethanol from KOPTEC. TMB substrate was obtained from Neogen (Enhanced KBlue TMB Substrate, Neogen Life Sciences, USA). Tween-20 was obtained in Fisher Scientific. Cortisol capture antibody (C-CAb), cortisol antigen (C) and cortisol-3-alkaline phosphatase (C-ALP) were acquired in Fitzgerald Industries. Insulin capture antibody (I-CAb) and HRP-labeled insulin detection antibody (HRP-DAb) were obtained from US Biological and human insulin standard (I) from Sigma-Aldrich. Human serum samples were obtained from the San Diego Blood Bank. Buffer solutions were prepared with Milli-Q water, e.g., PB (e.g., 0.1 M, pH 7.4), MES (e.g., 25 mM, pH 6.5).

Example Preparation Protocol of Thin Film Gold Sputtered Electrodes. The design and fabrication of the electrodes, as mentioned above, was carried out by the following example protocol. As a substrate for the manufacture of the electrodes, e.g., 0.76 mm thick PETG (glycol-modified polyethylene terephthalate) sheets were used. By means of a Cricut machine, a mask was designed cutting the laminated protective cover of the substrate. First, a Cr layer was deposited onto the polymer substrate in order to improve the adhesion of the subsequent metal to be deposited by sputtering, e.g., using a Denton Discovery 18 Sputter System under direct current (DC) mode at 200 W for 20 s, under Ar gas pressure of 2.4 mTorr. Then, a gold layer was deposited by using the same sputtering device under direct current (DC) mode at 200 W for 5 min, under Ar gas pressure of 2.4 mTorr. Finally, two 10-minute washes were performed to clean the electrodes by immersing it, first in isopropyl alcohol and then in water.

Example Protocol for Modifying Electrodes of a Dual C/I Sensor Chip. After cleaning the electrodes, the electrodes were dried with a compressed air gun and immersed in a SAM mixture solution including MUA (0.1 mM) and MCH (1.0 mM), e.g., overnight (4° C. and humidity conditions). The SAM modified electrodes were washed by immersion in pure ethanol and water, during 10 min each. The following steps were performed at room temperature and humidity conditions by drop casting with 2.5 μL on the surface of each working electrode with the appropriate solution according to the immunoassay. After each incubation step, three washes are performed by rinsing with PB (0.1 M, pH 7.4) and drying carefully with a compressed air gun.

The generated carboxylic groups in the electrode surface were activated by incubation in a mixture solution of EDC (0.4 M) and NHS (0.1 M) prepared in MES buffer (0.25 mM, pH 6.5) for 35 min. Then, the electrodes were incubated in the capture antibody solution 200 mL⁻¹ (30 min) or 100 μg mL⁻¹ (45 min) for cortisol or insulin immunoassay respectively (both prepared in PB 0.1 M, pH 7.4), followed by a 30 min blocking step of the remaining free activated carboxylic groups, with ethanolamine solution 2.0 M for cortisol and 1.0 M for insulin immunoassay (both prepared in 1:1 mixture of H₂O and NaOH (0.25 mM) checking the 8-9 basic pH). After this step, the electrodes can be stored in humidity conditions and 4° C. until use.

In the 20 min analysis step, depending on the type of detection (individual or simultaneous), the incubation was performed by placing a 2.5-4, drop on the corresponding working electrode (individual) or covering the two working electrodes with a 10-μL drop (simultaneous). In the individual detection, the WE was exposed to a mixture solution containing cortisol and cortisol-ALP (1:10 diluted) for C immunoassay, and, in the case of I immunoassay, a mixture containing insulin and HRP-DAb (1 μg mL⁻¹). For simultaneous detection, the 10-μL incubation drop contains the necessary reagents for both immunoassays.

After this step, the electrodes were washed first three times with SDS solution (e.g., 0.05% SDS in PB 0.1 M, pH 7.4), and subsequently three times with PB (0.1M, pH 7.4). Finally, the chips were dried using a compressed air gun. The chronoamperometric measurements were preformed sequentially, first the cortisol immunoassay and then the insulin immunoassay, applying +0.4 V or −0.1 V for 90 seconds, respectively. The cortisol detection was based on ALP/1-NPP (5.0 mM) system, which used 1 min hydrolysis step, whereas insulin detection employ TMB/HRP system.

FIGS. 11A-11C show diagrams illustrating example dual biomarker sensor chip devices and example techniques to implement the example dual-enzyme-based amperometric immunosensor chip for the simultaneous detection of cortisol and insulin biomarkers.

FIG. 11A shows images of the example Au-sputtered three-electrode system-based chip array on PETG substrate and a single sensor chip employed for dual C/I biosensor fabrication. The design of the dual C/I sensor chip relies on the example three-Au-electrode system, whose fabrication included the deposition of a gold thin-film by sputtering process on a plastic PETG substrate, obtaining an array 1100 of the dual C/I sensor chip devices 1150.

FIG. 11B shows a diagram illustrating the ALP/HRP multi-enzymatic labeling based C/I dual sensor chip 1150 comprising two Au working electrodes (WE) modified with the bioreceptors for the cortisol and insulin immunosensors, respectively, and a third Au electrode in the middle that acts as CE/RE. As shown in FIG. 11B, the sensor device 1150 includes two gold working electrodes (WE) in which by SAM functionalization-based attachment of the specific bioreceptors the detection of cortisol and insulin are localized, and a third electrode in the middle acting as reference/counter (RE/CE).

FIG. 11C shows a diagram depicting the immunoreaction and transduction processes involved in the C/I sensing. Cortisol and insulin detection are based on a competitive and a sandwich format immunoreaction, respectively, performed in a single simultaneous incubation step in which the sample is spiked with the HRP-labeled anti-insulin Ab and ALP-labeled cortisol reagents. After 20 min sample incubation, first cortisol detection is performed through amperometric measurement at +0.4V on Au WE1 through the oxidation of the phenolic compound obtained as a product of the phosphate aromatic derivative hydrolysis catalyzed by the captured ALP tag. Insulin is monitored on Au WE2 at −0.1V by recording the cathodic current resulting of the activity of the HRP tag after washing the chip surface and adding the TMB/H₂O₂ redox probe.

The diagram of FIG. 11C illustrates the heterogeneity of the immunoassay format and enzymatic labeling that the dual sensor chip relies on for simultaneous detection of both analytes. The cortisol sensor approach is based on a competitive immunoassay format using alkaline phosphatase (ALP) as enzymatic label of the tagged cortisol antigen (C-ALP) and 1-naphthyl phosphate (1-NPP) as the substrate for the detection reaction catalyzed/initiated by this hydrolase enzyme, while the insulin sensor is based on a sandwich immunoassay using horseradish peroxidase (HRP) as enzymatic label and 3,3′,5,5′-tetramethylbenzidine (TMB)/H₂O₂ as the mediator/substrate detection system. In these example implementations, the simultaneous detection of both biomarkers, which occurs within less than 25 min with the dual C/I sensor chip 1150, performed the incubation of a microliter sample supplemented with a known concentration of the enzyme tagged immunoreagents in one single 20 min-step, in which the C-ALP and the cortisol (C) are recognized by the anti-cortisol capture antibody in a competitive assay and the insulin (I) antigen is sandwiched by the HRP-labeled detector anti-insulin antibody and the immobilized anti-insulin capture antibody, respectively, in each sensing platform. In these example implementations, the extent of the affinity reactions was detected by amperometry in a judiciously optimized sequential transduction protocol, in which cortisol detection is first carried out followed by insulin detection. The example ALP-based amperometric detection of cortisol relies on the irreversible oxidation on the electrode surface of the enzymatic product 1-naphthol (1-NPL) at +0.4V obtained after catalytic hydrolysis of 1-NPP for 1 min, providing an anodic current that is directly proportional to the captured ALP-tag and, therefore, inversely proportional to the concentration of cortisol in the sample. In the case of the example HRP-approached insulin sensor, the amperometric transduction relied on the HRP/H₂O₂/TMB redox probe so that the cathodic current measured is due to the HRP catalyzed H₂O₂ reduction mediated by TMB where the oxidized form of the TMB is reduced at −0.1V on the electrode surface. Thus, the insulin concentration is directly proportional to the recorded current as it depends on the amount of the captured HRP tag.

FIG. 11D shows a diagram illustrating the method 1160 for fabricating an example embodiment of the C/I sensor chip 1150. The method 1160 includes a process 1161 for SAM formation on the electrodes of an unmodified sensor chip 1101 to produce a SAM-modified sensor chip 1103. The method 1160 includes a process 1163 for modification of functional groups on the SAM-modified sensor chip 1103 to produce a functionalized-SAM-modified sensor chip 1105. The method 1160 includes a process 1165 for immobilizing capture probes and/or produce a sensing layer on the functionalized-SAM-modified sensor chip 1105 to produce a pre-modified sensor chip 1107. The method 1160 includes a process 1167 for modification of the immobilized capture probes and/or sensing layer on the pre-modified sensor chip 1107, e.g., by implementing a blocking step for blocking the remaining free activated carboxylic groups on the modified electrodes, and thereby produce a C/I sensor chip 1150.

Example Electrochemistry-related Cross-talk Evaluation and Performance Optimization Towards Dual C/I Heterogeneous Enzyme Immunoassay. In order to attain high sensitivity in the detection of both biomarkers using the dual immunosensor chip, experimental variables that affect the analytical performance through electrochemical transductions and immunoreactions involved were thoroughly examined.

ALP is a hydrolase enzyme able to transform ortophosphoric monoesters into the inorganic phosphate ion and the corresponding alcohol products which has been widely used as a tracer in immunosensors due to its adaptability to electrochemical methods and suitability for generating an electroactive product from a non-interfering electroinactive substrate. As is well-known, this enzyme is characterized by exhibiting the highest catalytic activity and stability under alkaline conditions. As ALP has a broad substrate specificity, various compounds have been reported as enzyme substrates. Of these, 1-naphthyl phosphate (1-NPP) has been chosen in our protocol as it offers advantages including no electrochemical response in the conventional potential range, low oxidation potential of its enzymatic hydrolysis product, great sensitivity, and high stability.

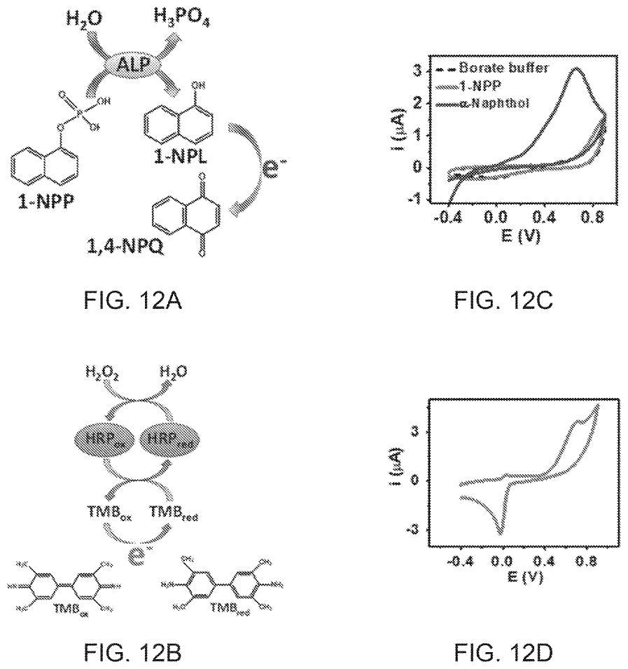

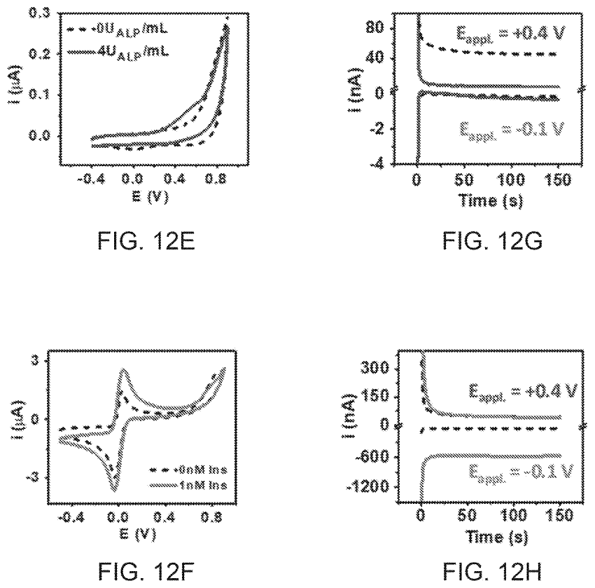

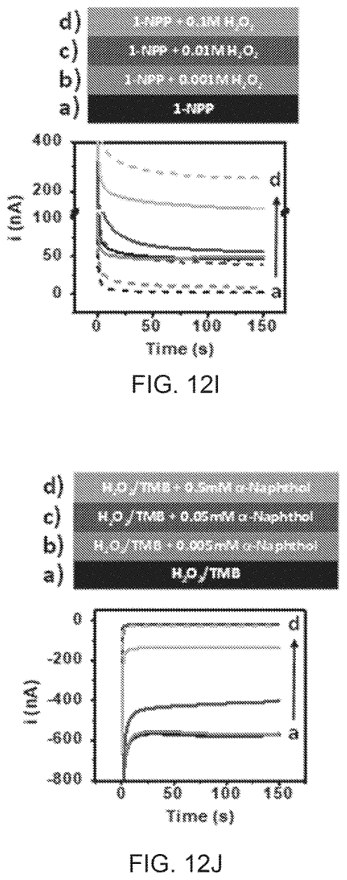

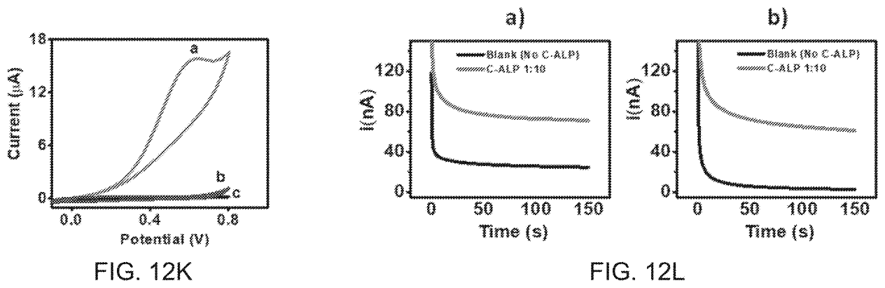

FIGS. 12A-12J shows illustrations and data plots depicting an example implementation for evaluating cross-talk during electrochemical transduction for cortisol-insulin dual analyte microchip electrodes of the example CA sensor chip device 1150. FIGS. 12A and 12B show schematic representations of ALP- (FIG. 12A) and HRP-mediated (FIG. 12B) enzymatic reactions. FIG. 12C shows a data plot depicting the CVs of 1-NPP and α-naphthol in borate buffer solution (pH 9.5) containing 5 mM Mg²⁺ ions (i) along with the CVs recorded for 1-NPP on the cortisol immunosensor in absence and presence of ALP-cortisol conjugate (ii). FIG. 12D shows a data plot depicting the CV of the commercial TMB/H₂O₂ on the bare Au microelectrode (i) and CVs of insulin immunosensor incubated in 0 and 1 nM insulin concentration and 1 μg mL⁻¹ of HRP-Ab conjugate (ii). The operating potentials for cortisol and insulin detection were shown as +0.4V and −0.1V, respectively. FIG. 12E shows (i) the amperometric response of the cortisol sensor chip incubated with 0 (dashed curve) and 100 ng·mL⁻¹ cortisol (blue curve) recorded under different potentials of +0.4 V and −0.1 V. C-ALP was 10 times diluted; and (ii) the amperometric response of the cortisol microchip to 0 (dashed curves) and 100 ng·mL⁻¹ cortisol (solid curves) obtained in different mediator solutions of TMB/H₂O₂ (black) and 1-NPP containing varying levels of TMB/H₂O₂. FIG. 12F shows (i) the amperometric response of the insulin immunosensor incubated with 0 (dashed curve) and 1 nM insulin (red curve) recorded under different potentials of +0.4 V and −0.1 V (e.g., where the concentration of HRP-Ab was 1 μg mL⁻¹) and (ii) the amperometric response of the insulin microchip to 0 (dashed curves) and 1 nM insulin (solid curves) obtained in different mediator solutions of TMB/H₂O₂ (black) and TMB/H₂O₂ containing varying levels of 1-NPP.

FIG. 12A shows the reaction sequence involved in transducing the cortisol immunoassay. 1-NPP hydrolyzes on the reaction with ALP, giving rise to the electroactive product 1-naphthol which oxidizes to 1,4-naphthoquinone on the electrode surface. At the same time, in the case of I sensor, the current is the electrocatalytic response of the TMB/H₂O₂ couple serving as cosubstrate to HRP enzyme tag. The electrocatalytic reaction involved in the insulin transduction is shown in FIG. 12B, in which the HRP catalyzes the reduction of H₂O₂ with the concurrent oxidation of TMB oxidation to TMB⁺ continued by reduction of such oxidized form on the electrode surface. Buffers presenting a Tris-HCl type molecular structure would be preferred as they promote transphosphorylation of the substrates. However, the optimum buffer should show low background currents besides possessing high oxidation limit and supporting product stability. Considering that the example sensor chip integrates a counter/pseudo-reference gold electrode, the electrochemical properties of different buffers through exploring their catalytic activity and possible interference for 1-NPL oxidation monitoring on an Au/Au 2-electrode system was assessed.

For this example implementation, whose example, experimental results are displayed in FIG. 12K, the current intensities measured by cyclic voltammetry (CV) on bare gold electrodes for Tris-HCl and borate buffers at alkaline pH 9.5 were compared. These experiments revealed that the Tris-HCl exhibits a large catalytic current whereas the borate buffer is noncatalytic. Additionally, the amperometric readout at +0.4V measured with the C sensor in the transduction of the 1-NPP reaction performed in such buffers in the presence and absence of C-ALP were also compared (see FIG. 12L), obtaining in agreement with the CV results a lower background current when borate buffer is used and hence providing a wider current window for sensitive detection of different cortisol concentrations.

FIGS. 12K and 12L show data plots of example results for evaluation of the buffer employed in ALP-based electrochemical transduction. FIG. 12K shows a data plot depicting cyclic voltammetry curves measured by exposing the example bare gold sputtered chip to (a) 5 mM Tris-HCl buffer pH 9.5 containing 5 mM MgCl2; (b) 5 mM borate buffer pH 9.5 containing 5 mM MgCl2; and (c) deionized water. FIG. 12L shows two data plots depicting chronoamperometric responses measured in the absence and presence of C-ALP 1:10 diluted with anti-cortisol antibodies modified sensor chips employing (a) Tris-HCl and (b) borate buffers containing 5 mM MgCl2 for the transduction reaction through 1-NPP substrate.

The electrochemical behavior of 1-NPP and its hydrolysis product, 1-naphthol, in borate buffer was examined by CV at the bare Au electrode, as shown in the data plot of FIG. 12C. For example, 1-NPP was not electrochemically active over the potential range studied but 1-naphthol displayed an irreversible chemical reaction due to its oxidation to the insoluble quinoid product. Moreover, CV measurements performed with the C sensor in absence and presence of C-ALP exposed the specificity of the transduction reaction since the oxidation of the alcohol derivative generated only in the presence of ALP is observed, as shown in the data plot of FIG. 12E. Similarly, for example, the CV of the commercial TMB/H₂O₂ was carried out on the bare Au electrode (see, e.g., FIG. 12D), revealing solely the characteristic H₂O₂ reduction peak obtained at negative currents since in the absence of HRP the TMB oxidation by H₂O₂ is a remarkably slow process. As illustrated in the CVs measured with the I sensor once the HRP-tagged sandwich is completed (see, e.g., FIG. 12F), the reversible TMB oxidation reaction is produced in the detection of the H₂O₂ catalyzed by HRP, so that the oxidation and reduction peaks occur at a suitable low potential which avoids possible interferences generated at potentials above +0.4V.

Additionally, the selectivity of the operating potentials applied for dual analyte measurements was demonstrated in FIGS. 12G and 12H, which illustrate the negligible response of cortisol and insulin immunosensors when the amperometric curves were measured at potentials of −0.1V for cortisol and +0.4V for insulin. Overall, the above observations from the example, experimental implementations suggest the initial tolerability of different applied potentials for selective amperometric detection of 1-NPL product (at +0.4V) and H₂O₂ substrate (at −0.1V) in an integrated dual chip without apparent electrochemical interference. However, the coexistence of these species in the transduction reactions characteristic of each sensor approach towards simultaneous detection was studied and characterized as explained below.

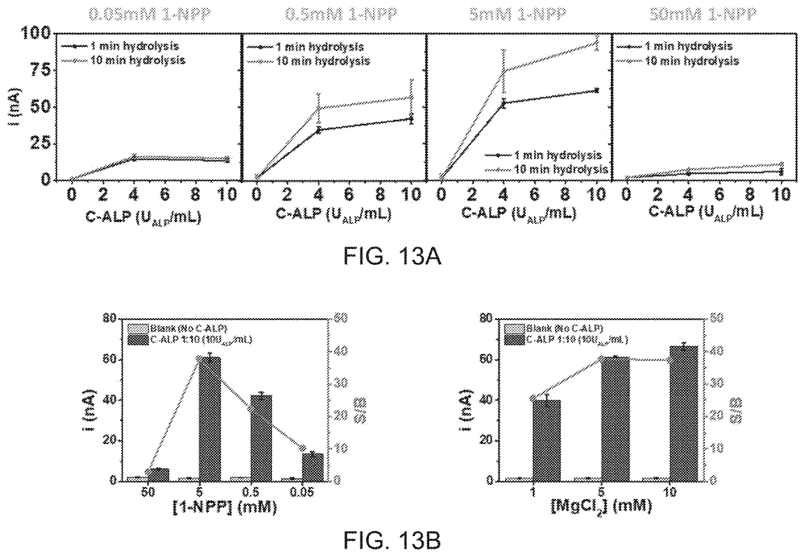

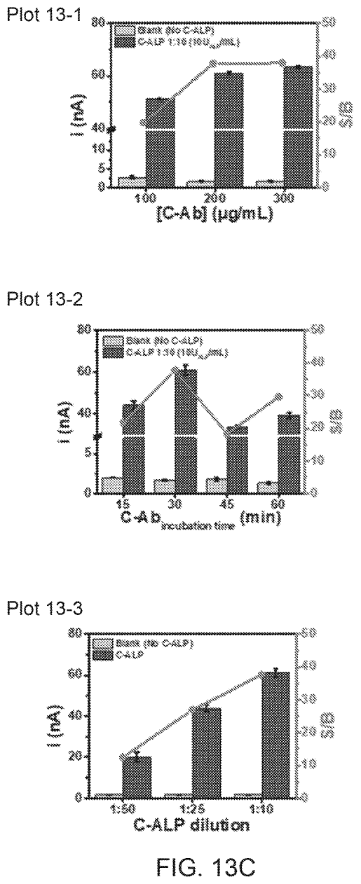

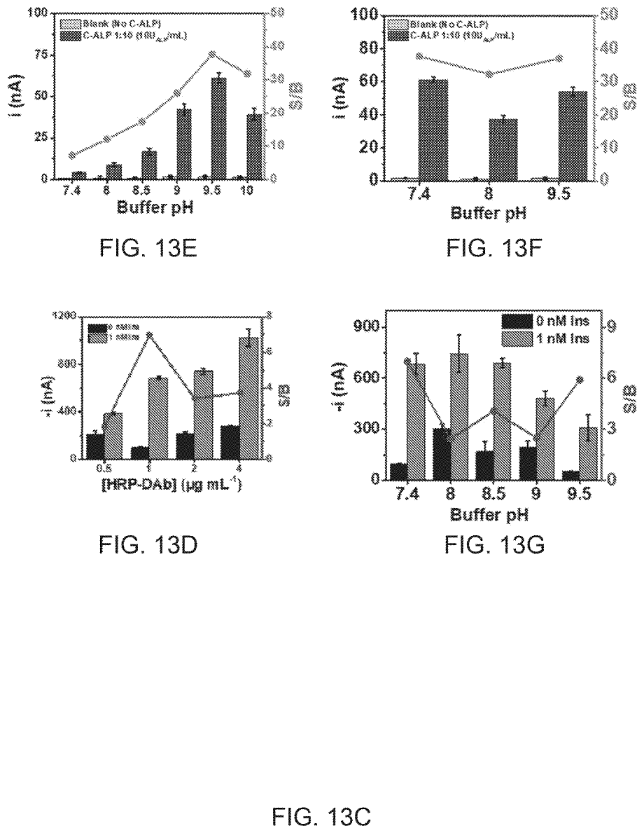

To develop highly sensitive immunosensing systems, various important parameters affecting the performance of the dual enzymatic reactions were investigated for optimization, for example, including buffer properties, reaction time and substrates concentration. Focusing on cortisol amperometric immunoassay, for these example implementations, the effect of parameters affecting the activity of the ALP was evaluated, such as concentration and hydrolysis time of 1-NPP substrate, and concentration of MgCl₂ as the presence of Mg²⁺ ions increases the hydrolysis rate of the enzyme substrate. For example, the longer hydrolysis time yielded the higher amperometric signal since a larger amount of 1-NPL to be oxidized on the electrode surface is generated, as shown in FIG. 13A. Nevertheless, since a dual biosensor chip able to detect both diabetes biomarkers in less than 25 min is highly desirable, a convenient hydrolysis time of 1 min was adopted. Furthermore, taking as the selection criterion the largest ratio between the amperometric responses measured in the presence and absence of C-ALP, a concentration of 5 mM was selected for both 1-NPP substrate and MgCl₂ (e.g., example results included in FIG. 13B).

FIGS. 13A and 13B show data plots depicting example results from evaluation of the experimental conditions for ALP-based electrochemical transduction. The data plots in FIG. 13A shows current intensity values recorded for different 1-NPP hydrolysis times and 1-NPP concentrations. The data plots in FIG. 13B shows the effect on measured amperometric response of the transduction solution composition considering 1-NPP and MgCl₂ concentrations.

Additionally for the C sensor, experimental variables related to the immunoreaction were optimized (see, e.g., FIG. 13C), such as anti-cortisol antibody concentration (e.g., 200 μg mL⁻¹), time required for the immobilization of this bioreceptor on the SAM-functionalized electrode surface (30 min), and C-ALP dilution (1:10). To accomplish these example experiments, a short assay time for the competition, and therefore for the sample incubation on the dual C/I chip, of only 20 min was established. In the case of the insulin sensor, the sensor platform modification protocol and transduction performance were applied, so that only the working conditions concerning sample incubation were adapted accordingly to the 20 min 1-step analysis for the novel application of the dual biosensor chip. Example experimental implementations of the HRP-labeled insulin antibody was performed (see, e.g., FIG. 13D), obtaining an optimal value of 1 μg mL⁻¹.

FIGS. 13C and 13D show data plots depicting example results for an example optimization of experimental variables affecting immunoassay performance. The data plots of FIG. 13C show example results studying cortisol biosensor optimization. The data plot of FIG. 13D shows example results of the optimization of the HRP-labeled detector Ab for insulin biosensor.

Considering that each sensor could present different optimal buffer pH for the biorecognition and transduction reactions, a comprehensive study to attain the most appropriate conditions that accommodate both immunoassay approaches towards simultaneous detection of C/I biomarkers with the biosensor chip was carried out. As can be seen in the results included in FIG. 13E, the highest amperometric response in presence of captured ALP-tagged cortisol antigen is recorded when the ALP catalyzed hydrolysis of the 1-NPP occurs in borate buffer at pH 9.5, and conversely, as expected, the minimal activity of the enzyme was observed when the hydrolysis reaction was performed at pH 7.4 as the phosphate buffer can lead to saturate the active sites of the ALP enzyme. Moreover, the effect of the pH on the sample incubation was examined for C sensor, obtaining that the extent to which the competition reaction occurs is almost the same at both pH 9.5 and pH 7.4 (see, e.g., FIG. 13F). Similarly, in the case of the sandwich based I assay, the feasibility of the sample incubation step in borate buffer was investigated as the HRP-based transduction is carried out by placing directly the commercial solution of H₂O₂/TMB on the electrode, observing no significative loss of sensitivity at pH 9.5 compared to pH 7.4 (see, e.g., FIG. 13G). The fact that the sample incubation on the dual C/I sensor chip could be realized in the same buffer pH conditions drastically simplified the analysis performance. Regarding the physiological pH close to 7.4, the phosphate buffer was selected as sample incubation medium for further studies on the dual sensor chip.

FIGS. 13E-13G show data plots depicting example results of an evaluation of the effect of the buffer pH on the amperometric responses. The data plot of FIG. 13E shows example results of a pH study for ALP-based transduction regarding the characteristics of the 1-NPP solution. The data plot of FIG. 13F shows example results of a pH study for cortisol immunoassay incubation. The data plot of FIG. 13G shows example results of a pH study for insulin immunoassay incubation.

In the example implementations, once the C/I immunoreaction protocol was defined, the cross-talk for the transduction procedures employed for the dual enzyme tags was explored towards possible simultaneous amperometric readout at cortisol and insulin electrodes. In FIG. 121 are displayed the current signals obtained for electrodes incubated with the blank and C-ALP conjugate (1:10 dilution) solutions in the presence of varying concentrations of H₂O₂. As can be seen, false higher amperometric responses were obtained in the presence of H₂O₂, which can be ascribed to the catalytic oxidation of 1-naphthol by H₂O₂. In the same way, the insulin detection performance was assessed in the presence of varying concentrations of 1-naphthol.

As shown by obtained results in FIG. 12J, 1-naphthol also affects negatively the current signal of the insulin sandwich immunoassay. This example data suggests that the HRP-mediated transduction reaction would be influenced by the reagents characteristic of the ALP-mediated reaction since the peroxidase also catalyzes the oxidation of 1-naphthol as well as the negative effect of the insoluble oxidation product 1,4-naphthoquinone generated that may passivate the electrode by accumulation of such electroinactive compound. Based on these example results, a sequential readout protocol for the enzymatic reactions was adapted for the example implementations. In this sense, the order in which the sequential electrochemical transduction can be carried out and the physical disposition of the 10 μL-drop of the corresponding enzyme substrate solution arranged on the dual chip for the detection of each analyte were evaluated.

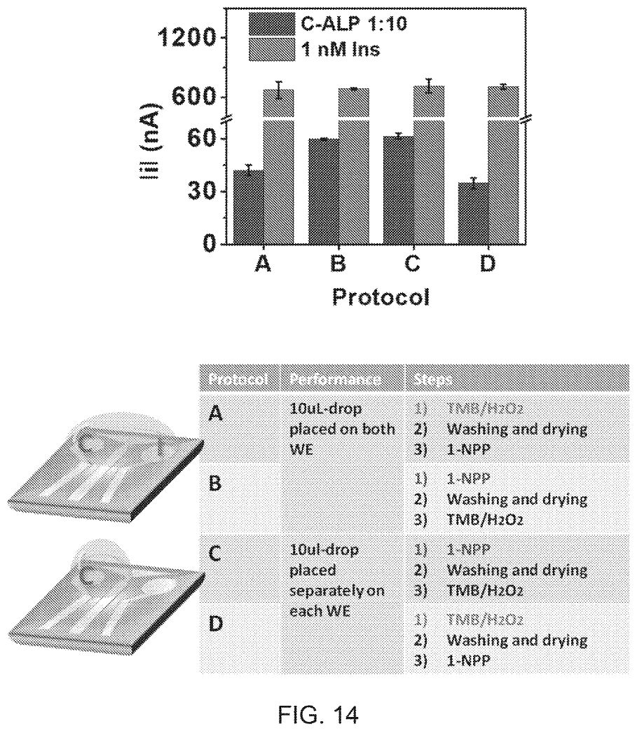

From the results obtained, shown in FIG. 14, it could be concluded that the ALP electrode reaction with 1-NPP for the measurement of cortisol should be performed first, followed after an essential washing step by the H₂O₂/HRP/TMB electrode reaction, regardless of whether the measurement solutions are located in the corresponding WE or covering both C-WE and I-WE, since in the case of applying the reverse order the signal of the C sensor is affected. This can be attributed to the fact that in the HRP reaction the TMB is oxidized, localizing mostly in the RE/CE gold electrode since a change to characteristic blue color corresponding to TMBₒₓ is observed, and then reduced on the electrode surface and this compound is known to adsorb and passivate gold electrodes, compromising the ALP-based transduction. It is worth mentioning that the optimized measurement protocol would enable a microfluidics-based automation of the dual biosensor chip.

FIG. 14 shows a data plot and a diagram depicting example results of an evaluation of an example sequential transduction protocol for the example dual C/I sensor chip, with the example protocol described in the table of the diagram.

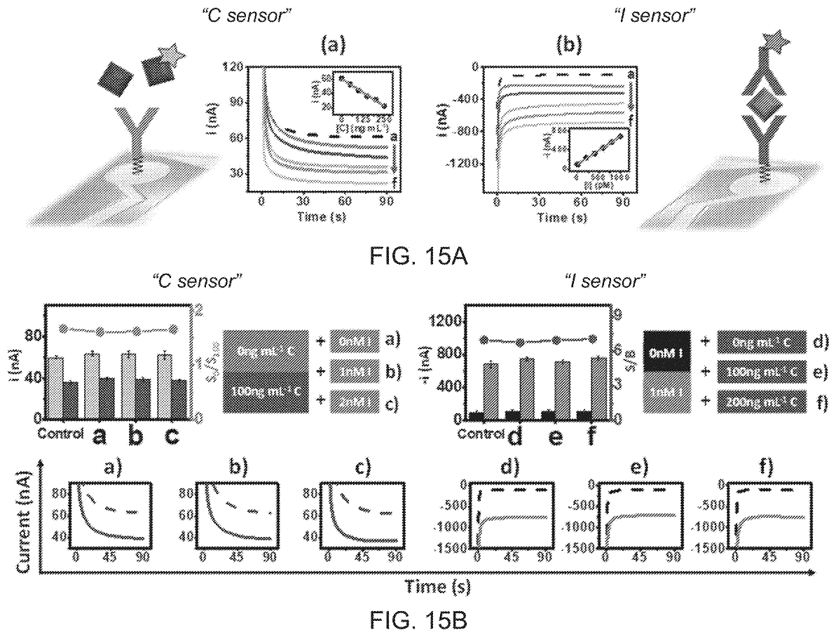

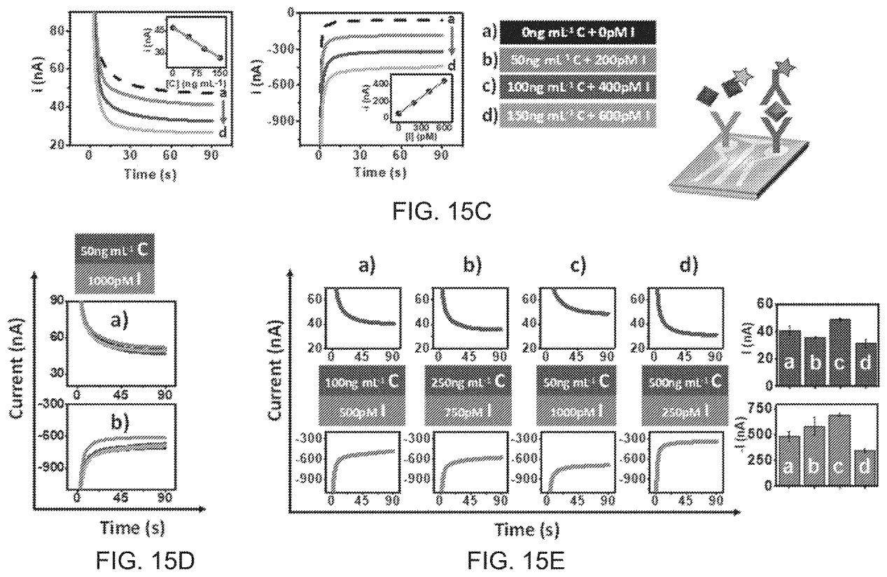

Example Analytical Performance of the Example C/I Sensor Chip. The electrochemical sensing performance and operational characteristics of the example dual C/I biosensor chip were evaluated toward rapid and sensitive simultaneous detection of cortisol and insulin biomarkers. The analytical performance of each immunoassay was first characterized individually by detecting increasing concentrations of cortisol and insulin solutions prepared in phosphate buffer in a minimum sample volume of 2.5 μL, e.g., obtaining the calibration curves shown in FIG. 15A. A linear behavior of the amperometric response with the cortisol concentration was observed for the C sensor in the range between 0 and 250 ng mL⁻¹, being the maximum signal corresponding to Ong mL⁻¹ the non-competition reference value. On the other hand, the sandwich format based I sensor was characterized for insulin concentrations ranged from 0 to 1 nM. Thus, the limits of detection (LOD) estimated from the calibration data were 13.4 ng mL⁻¹ and 30.6 pM for cortisol and insulin, respectively. These example results exhibited the capacity of the example dual C/I sensor to detect these biomarkers at the levels required in clinical samples.

FIGS. 15A-15E show illustrative diagrams and data plots depicting example results of an example implementation for evaluating electrochemical sensing performance of example C/I dual chip devices in phosphate buffer solution.

FIG. 15A shows an illustrative diagram alongside a data plot depicting the amperometric response of the example C sensor electrode (left illustration, data plot (a)), and of the example I sensor electrode (right illustration, data plot (b)) to increasing concentrations of cortisol from a) 0 to f) 250 ng mL⁻¹ with 50 ng mL⁻¹ increments, and insulin from a) 0 to f) 1000 pM with 200 pM increments, respectively. Insets of data plots (a) and (b) of FIG. 15A are the corresponding calibration plots.

FIG. 15B shows data plots depicting the immuno-reaction related cross-talk evaluation towards cortisol and insulin simultaneous detection. In these example implementations, amperometric signals were recorded with the example C sensor for 0 (grey dashed curves) and 100 ng mL⁻¹ cortisol (blue curves) in absence (control) and presence of mL⁻¹ of HRP-Anti-insulin antibody and a) 0 nM, b) 1 nM and c) 2 nM insulin. Amperometric signals were recorded with the example I sensor for 0 (black dashed curves) and 1 nM insulin (red curves) in absence (control) and presence of 1:10 diluted ALP-cortisol and a) 0 ng mL⁻¹, b) 100 ng mL⁻¹, c) 200 ng mL⁻¹ cortisol.

FIG. 15C shows data plots depicting amperometric responses obtained in the simultaneous detection of increasing concentrations of cortisol/insulin in a single sample droplet containing both biomarkers from (a) Ong mL⁻¹ cortisol+0 pM insulin (black curves) to (d) 150 ng mL⁻¹ cortisol+600 pM insulin (green curves), with increments of 50 ng mL⁻¹ and 200 pM for cortisol and insulin, respectively. Inset data plots are the corresponding calibration plots.

FIG. 15D shows data plots showing the reproducibility of 6 dual sensor chips in the measurement of a mixture containing a) cortisol 50 ng mL⁻¹ and b) insulin 1 nM.

FIG. 15E shows data plots depicting the amperometric responses obtained in the simultaneous detection of different mixtures of cortisol/insulin analytes whose concentrations range from 50 to 500 ng mL⁻¹ for cortisol (blue curves) and from 250 to 1000 pM for insulin (red curves).

In these example implementations, the potential cross-reactivity during the incubation of the sample toward simultaneous detection of both biomarkers was carefully examined for each sensor so that the amperometric signals measured in the absence and presence of a constant concentration of the target analyte itself were compared with those obtained in the measure of PBS solutions containing, besides the opposite assay enzyme-conjugated reagent, different concentrations of the complementary analyte. As can be seen in the example results displayed in FIG. 15B, no apparent effect was observed on the amperometric responses recorded in each biosensing platform, which supports the assay specificity and complete feasibility of the optimized 1-step analysis protocol.

In order to demonstrate the applicability of the fabricated C/I chips in the simultaneous detection of cortisol and insulin performed in microliter sample droplet, the measurement of PBS solutions of 104, containing increasing concentrations of both biomarkers was carried out. To accomplish this study, the applied analysis protocol implied first the incubation of the sample already supplemented with the HRP-labeled detector Ab and the ALP-labeled cortisol antigen onto the biosensor chip for 20 min. A sequential electrochemical detection protocol is then performed in which the cortisol transduction is first carried out involving 1 min of 1-NPP substrate hydrolysis and 1.5 min of signal measurement and subsequently the insulin transduction through the HRP/H₂O₂/TMB redox probe for 1.5 min, applying a washing step between them. Calibrations presented in FIG. 15C for both cortisol and insulin analytes illustrate the reliability and minimal cross-talk that characterizes such analysis protocol as clear response increments upon increasing target biomarker concentrations were obtained for all cases. Furthermore, the reproducibility of fabrication and functioning of the single-use dual biosensor chip was evaluated by testing six different dual C/I chips constructed following the same fabrication protocol in the measurement of a PBS solution containing 50 ng mL⁻¹ cortisol and 1000 pM insulin. Thus, the amperometric signals recorded shown in FIG. 15D yielded RSD values of 3.4% and 4.9% for cortisol and insulin detection, respectively. In addition, the dual biosensor chip was exposed to C-I mixture solutions with different varying concentrations ranging between 50 to 500 ng mL⁻¹ and 250 to 1000 pM for cortisol and insulin analytes, respectively. The amperometric responses obtained for cortisol (blue curves) and insulin (red curves) represented in FIG. 15E highlight the proven capability of the novel dual biosensor to perform simultaneous C/I detection with negligible cross-talk.

Applicability of the Dual C/I Chip in Serum Cortisol and Insulin Detection. The applicability of the biosensor chip in the direct detection of cortisol and insulin in untreated serum samples towards decentralized on-the-spot analysis of this biomarkers was explored, as this is a very valuable biological specimen in diabetic patients monitoring whose matrix excludes blood cells and clotting factors that could interfere with the analysis, keeping the biomolecules of interest in its composition. The analytical performance of the C/I sensors in the detection of cortisol and insulin in such matrix was first individually evaluated by placing onto each biosensing platform a 2.5 μL sample droplet of an undiluted serum sample purchased from the SDBB spiked with the corresponding assay enzymatic tracer and increasing concentrations of the target biomarker, ranging from 0 to 400 ng mL⁻¹ for cortisol and 0 to 4 nM for insulin.

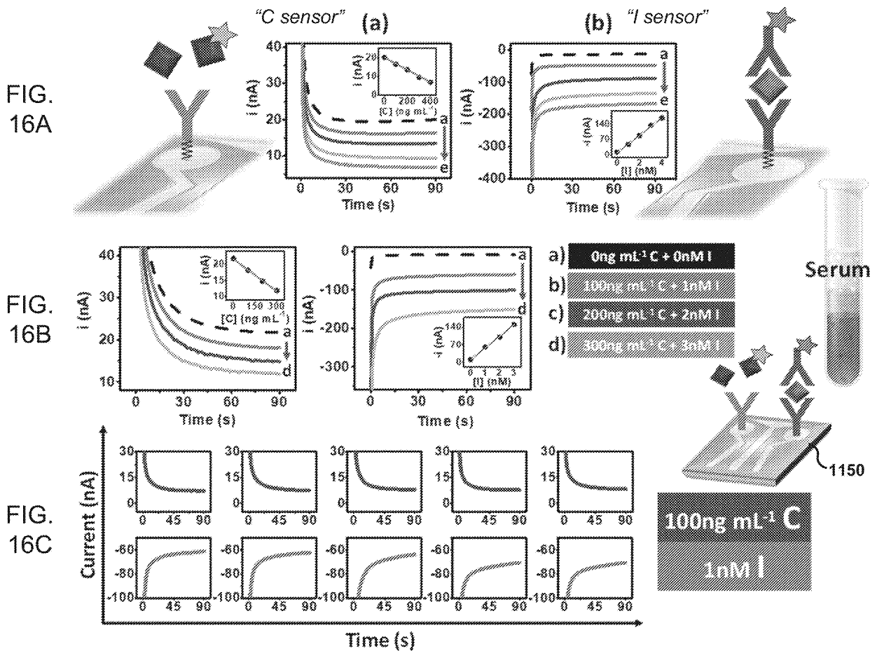

FIGS. 16A-16C show data plots and accompanying illustrations depicting the electrochemical sensing performance of example C/I dual chips in non-treated human serum. FIG. 16A shows the amperometric response of the example C sensor electrode (left illustration, data plot (a)) and of the example I sensor electrode (right illustration, data plot (b)) to increasing concentrations of cortisol from a) 0 to e) 400 ng mL⁻¹ with 100 ng mL⁻¹ increments, and insulin from a) 0 to e) 4 nM with 1 nM increments, respectively. The insets are the corresponding calibration plots.

FIG. 16B show data plots depicting the amperometric responses obtained in the simultaneous detection of increasing concentrations of cortisol/insulin in a single serum sample droplet containing both biomarkers from (a) Ong mL⁻¹ cortisol+0 pM insulin (black curves) to (d) 300 ng mL⁻¹ cortisol+3 nM insulin (green curves), with increments of 100 ng mL⁻¹ and 1 nM for cortisol and insulin, respectively. The insets are the corresponding calibration plots. FIG. 16B also shows an illustration depicting a test tube having the serum inside, from which a sample can be deposited on the example C/I sensor chip device 1150.

FIG. 16C shows data plots depicting the reproducibility of the example dual C/I sensor chip in the measurement of a mixture containing cortisol 100 ng mL⁻¹ (blue curves) and insulin 1 nM (red curves).

In both cases of the example implementations, the value of the amperometric responses were lower than those obtained in the buffer matrix. However, despite this sensitivity loss due to the sample matrix, C/I sensors demonstrated adequate performance towards simultaneous detection of both serum analytes in a minimum sample droplet as can be deduced from the LOD values reached, corresponding to 23.1 ng mL⁻¹ and 177 pM for cortisol and insulin, respectively, covering thus clinically relevant concentration range of these biomarkers in this blood matrix. Additionally, in order to assess the viability of simultaneous detection by exposing the C/I biosensor chip to a 10 μL-drop of untreated serum, dual calibrations were measured in serum samples containing increasing concentrations of both analytes, ranging from 0 to 300 ng mL⁻¹ for cortisol and from 0 to 3 nM for insulin. As can be observed in the amperometric curves shown in FIG. 16B, convenient 100 ng mL⁻¹ and 1 nM concentration increments measurements for cortisol and insulin, respectively, were provided by the dual chip in less than 25 min, so that the same performance regarding the selective biorecognition processes and subsequent electrochemical transduction as in the individual detection was obtained, as well as a negligible effect on the dual assay sensitivity of a higher sample volume for serum analysis.

Further, the reproducibility yielded in the measurement of a serum sample containing 100 ng mL⁻¹ cortisol and 1 nM insulin realized with five different example C/I dual chips demonstrated the robustness and reliability of the fabrication and analytical protocols optimized for the novel biosensor device as verified in the RSD values obtained for cortisol measurements (5.1%) and insulin (7.1%).

The example results from the above-discussed experimental implementations of the example C/I sensor chip device demonstrates the first disposable dual electrochemical sensor chip for simultaneous detection of cortisol and insulin in which the different immunoassay approaches based on the use of competitive and sandwich formats and fundamentally distinct enzymatic labeling strategies for amperometric readout given by the ALP and HRP have been implemented. To achieve its successful performance in the dual detection of these two biomarkers at significantly different concentration levels on a single chip, the analytical protocol was critically optimized by addressing potential cross-talk due to the heterogeneous nature of the bioassays involved. The dual C/I biosensor chip has demonstrated very attractive performance towards sensitive and direct detection of cortisol and insulin in relevant body fluids for diabetes monitoring such as blood serum, carried out on a microliter sample drop. Moreover, the example C/I sensor chip represents a promising technology for screening and diagnosis through the detection of protein biomarkers linked to diabetes and other diseases.

### Examples

The following examples are illustrative of several embodiments of the present technology. Other exemplary embodiments of the present technology may be presented prior to the following listed examples, or after the following listed examples.

In some embodiments in accordance with the present technology (example A1), a sensor device for monitoring one or more analytes includes a substrate; and one or more detecting electrodes disposed on the substrate, the one or more detecting electrodes including one or both of (i) a first electrode to sense an analyte by an enzymatic or direct electrochemical detection, and (ii) a second electrode to sense the analyte or a different analyte by an immunosensing detection; and a counter electrode to detect a signal with respect to that detected by the one or both of the first electrode and the second electrode.

Example A2 includes the device of any of examples A1-A4, wherein the one or more analytes include glucose and/or insulin.

Example A3 includes the device of any of examples A1-A4, wherein the one or more analytes is contained in a fluid deposited on the sensor, the fluid including saliva, blood, tears, interstitial fluid, or combination thereof.

Example A4 includes the device of any of examples A1-A3, wherein the substrate is flexible, bendable and/or stretchable.

In some embodiments in accordance with the present technology (example A5), a sensor device for monitoring glucose and insulin includes a substrate; and a plurality of electrodes disposed on the substrate, the plurality of electrodes including a first electrode to sense glucose, a second electrode to sense insulin, and a counter electrode to the first and second electrodes, wherein the first electrode includes a glucose oxidase enzyme linked to a surface of the first electrode, and the second electrode includes an insulin capture antibody linked to a second electrode through a self-assembly monolayer, and wherein, when the device is electrically coupled to an electronics unit, the device is operable to detect insulin and glucose from a fluid.

Example A6 includes the device of any of examples A5-A10, wherein the fluid is saliva, blood, tears, interstitial fluid, or combination thereof.

Example A7 includes the device of any of examples A5-A10, wherein the substrate is flexible.

Example A8 includes the device of any of examples A5-A10, wherein the first electrode and second electrodes are gold.

Example A9 includes the device of any of examples A5-A10, wherein the counter electrode is silver/silver chloride (Ag/AgCl)

Example A10 includes the device of any of examples A5-A9, wherein the substrate is polyethylene terephthalate (PETG), polyethylene naphthalate (PEN), polyimide (PI), or any combination therefore.

In some embodiments in accordance with the present technology (example A11), a method of detecting insulin and glucose in a fluid on a single sensor includes contacting a fluid sample with a sensor device comprising a substrate comprising a first electrode for sensing glucose, a second electrode for sensing insulin, and a counter electrode; and measuring a parameter associated with the glucose or the insulin, or both.

Example A12 includes the method of any of examples A11-A21, wherein the measuring the parameter associated with the glucose and the insulin is measured simultaneously.

Example A13 includes the method of any of examples A11-A21, further comprising determining a concentration of the glucose or the insulin, or both, wherein the concentration of the glucose and/or the insulin is determinable from a single drop of the fluid.

Example A14 includes the method of any of examples A11-A21, wherein the concentration of glucose and insulin can be determined from a 10 μL sample in less than 20 minutes after the fluid sample contacts the substrate.

Example A15 includes the method of any of examples A11-A21, wherein the fluid includes saliva, blood, tears, interstitial fluid, or combination thereof.

Example A16 includes the method of any of examples A11-A21, wherein the concentration insulin and glucose can be detected at physiological levels.

Example A17 includes the method of any of examples A11-A21, wherein the insulin is detected at pico molar (pM) concentrations and/or the glucose is detected at milli molar (mM) concentrations.

Example A18 includes the method of any of examples A11-A21, determining a parameter relevant to a clinical diagnoses of diabetes in a subject based on the concentration of glucose and insulin measured in a fluid obtained from the subject.

Example A19 includes the method of any of examples A11-A21, wherein the first electrode of the sensor device includes a glucose oxidase enzyme linked to a surface of the first electrode, and the second electrode includes an insulin capture antibody linked to a second electrode through a self-assembly monolayer.

Example A20 includes the method of any of examples A11-A21, wherein the first electrode is configured for sensing one or both of cortisol and ketone bodies.

Example A21 includes the method of any of examples A11-A20, comprising determining a concentration of the glucose, insulin, ketone bodies, cortisol or any combination thereof, wherein the concentration of the glucose, insulin, ketone bodies, and/or cortisol is determinable from a single drop of the fluid.

In some embodiments in accordance with the present technology (example B1), a sensor device for simultaneous monitoring two or more analytes includes a substrate; and two or more detecting electrodes disposed on the substrate, the two or more detecting electrodes including a first electrode to sense a first analyte by an enzymatic or direct electrochemical detection, and a second electrode to sense a second analyte by an immunosensing detection; a first functionalization layer disposed on the first electrode, the first functionalization layer including a catalyst to facilitate an electrochemical reaction to detect the first analyte at the first electrode; a second functionalization layer disposed on the second electrode, the second functionalization layer including a capture antibody to facilitate an electrochemical immune assay reaction to detect the second analyte at the second electrode; and a counter electrode to detect a signal with respect to that detected by the one or both of the first electrode and the second electrode.

Example B2 includes the device of any of examples B1-B20, wherein the first analyte includes glucose and the second analyte includes insulin.

Example B3 includes the device of any of examples B1-B20, wherein the first analyte includes cortisol and the second analyte includes insulin.

Example B4 includes the device of any of examples B1-B20, wherein the first analyte and the second analyte are contained in a first fluid or are separately contained in the first fluid and a second fluid, wherein the first fluid and the second fluid include one of saliva, blood, tears, interstitial fluid, or a combination thereof.

Example B5 includes the device of any of examples B1-B20, wherein the capture antibody is attached to the second electrode via a self-assembled monolayer (SAM).

Example B6 includes the device of any of examples B1-B20, wherein the second analyte includes insulin and the capture antibody includes an anti-insulin antibody, and wherein the second functionalization layer is configured to facilitate a sandwich electrochemical immunoassay to detect a concentration of insulin in a biofluid.

Example B7 includes the device of any of examples B1-B20, wherein the sandwich electrochemical immunoassay includes horseradish peroxidase (HRP) attached to a detection antibody used as an enzyme label and 3,3′,5,5′-tetramethylbenzidine (TMB)/hydrogen peroxide (H₂O₂) used as an enzymatic substrate/mediator for detection of the insulin after covalent attachment of the insulin to the capture antibody and subsequent binding of the detection antibody.

Example B8 includes the device of any of examples B1-B20, wherein the concentration of the insulin is detectable within a picomolar concentration range by the sensor device.

Example B9 includes the device of any of examples B1-B20, wherein the catalyst includes glucose oxidase (GOx).

Example B10 includes the device of any of examples B1-B20, wherein the GOx is immobilized inside a permeable polymer film.

Example B11 includes the device of any of examples B1-B20, wherein the permeable polymer film comprises a tetrathiafulvalene (TTF) mediator layer formed on the first electrode and a layer comprising GOx, chitosan, and a sulfonated tetrafluoroethylene polymer.

Example B12 includes the device of any of examples B1-B20, wherein the first analyte and the second analyte each include one diabetic biomarker selected from a group consisting of glucose, lactate, ketone bodies, insulin, cortisol, and C-peptide.

Example B13 includes the device of any of examples B1-B20, comprising an electronics unit including an electric circuit coupled to a data processing unit, wherein, when the sensor device is electrically coupled to the electronics unit, the device is operable to detect the first analyte and the second analyte and determine a concentration of the first analyte and of the second analyte.

Example B14 includes the device of any of examples B1-B20, wherein the first electrode and second electrodes include gold.

Example B15 includes the device of any of examples B1-B20, wherein the counter electrode includes silver/silver chloride (Ag/AgCl).

Example B16 includes the device of any of examples B1-B20, wherein the first electrode and the second electrode are configured on opposing sides of the counter electrode.

Example B17 includes the device of any of examples B1-B20, wherein the two or more detecting electrodes further include a third electrode to sense a third analyte by the enzymatic or direct electrochemical detection or by the immunosensing detection.

Example B18 includes the device of any of examples B1-B20, wherein the substrate includes a plastic.

Example B19 includes the device of example B18, wherein the plastic includes polyethylene terephthalate (PET), polyethylene terephthalate glycol (PETG), polyethylene naphthalate (PEN), or polyimide (PI).

Example B20 includes the device of any of examples B1-B19, wherein the substrate is flexible, bendable and/or stretchable.

In some embodiments in accordance with the present technology (example B21), a method for simultaneously detecting multiple analytes on a single sensor platform includes contacting a fluid sample with a sensor device comprising a substrate and a plurality of electrodes disposed on the substrate, the electrodes including a first electrode for sensing a first analyte, a second electrode for sensing a second analyte, and a counter electrode; and measuring a parameter associated with the first analyte or the second analyte, or both.

Example B22 includes the method of any of examples B21-B30, wherein the first analyte includes glucose and the second analyte includes insulin.

Example B23 includes the method of any of examples B21-B30, wherein the first analyte includes cortisol and the second analyte includes insulin.

Example B24 includes the method of any of examples B21-B30, wherein the first analyte and the second analyte each include one diabetic biomarker selected from a group consisting of glucose, lactate, ketone bodies, insulin, cortisol, and C-peptide.

Example B25 includes the method of any of examples B21-B30, wherein the first analyte and the second analyte are contained in a first fluid sample contacted on the sensor device or the first analyte and the second analyte are separately contained in the first fluid sample and a second fluid sample where the method comprises contacting the second fluid sample on the sensor device after the contacting the first fluid sample, and wherein the first fluid sample and the second fluid sample include one of saliva, blood, tears, interstitial fluid, or a combination thereof.

Example B26 includes the method of any of examples B21-B30, further including determining a concentration of the first analyte or the second analyte, or both, wherein the concentration of the first analyte or the second analyte, or both is determinable from a single drop of the fluid.

Example B27 includes the method of any of examples B21-B30, wherein the concentration of the first analyte or the second analyte, or both can be determined from a 10 μL sample in less than 20 minutes after the fluid sample contacts the sensor device.

Example B28 includes the method of any of examples B21-B30, wherein the first analyte includes glucose and the second analyte includes insulin, and wherein the insulin is detected at a pico molar (pM) concentration range and the glucose is detected at a milli molar (mM) concentration range.

Example B29 includes the method of any of examples B21-B30, comprising determining a parameter relevant to a clinical diagnoses of diabetes in a subject based on the concentration of glucose and insulin measured in a fluid obtained from the subject.

Example B30 includes the method of any of examples B21-B30, wherein the sensor device includes the device of any of examples B1-B20.

Implementations of the subject matter and the functional operations described in this patent document can be implemented in various systems, digital electronic circuitry, or in computer software, firmware, or hardware, including the structures disclosed in this specification and their structural equivalents, or in combinations of one or more of them. Implementations of the subject matter described in this specification can be implemented as one or more computer program products, i.e., one or more modules of computer program instructions encoded on a tangible and non-transitory computer readable medium for execution by, or to control the operation of, data processing apparatus. The computer readable medium can be a machine-readable storage device, a machine-readable storage substrate, a memory device, a composition of matter effecting a machine-readable propagated signal, or a combination of one or more of them. The term “data processing unit” or “data processing apparatus” encompasses all apparatus, devices, and machines for processing data, including by way of example a programmable processor, a computer, or multiple processors or computers. The apparatus can include, in addition to hardware, code that creates an execution environment for the computer program in question, e.g., code that constitutes processor firmware, a protocol stack, a database management system, an operating system, or a combination of one or more of them.

A computer program (also known as a program, software, software application, script, or code) can be written in any form of programming language, including compiled or interpreted languages, and it can be deployed in any form, including as a stand-alone program or as a module, component, subroutine, or other unit suitable for use in a computing environment. A computer program does not necessarily correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

The processes and logic flows described in this specification can be performed by one or more programmable processors executing one or more computer programs to perform functions by operating on input data and generating output. The processes and logic flows can also be performed by, and apparatus can also be implemented as, special purpose logic circuitry, e.g., an FPGA (field programmable gate array) or an ASIC (application specific integrated circuit).

Processors suitable for the execution of a computer program include, by way of example, both general and special purpose microprocessors, and any one or more processors of any kind of digital computer. Generally, a processor will receive instructions and data from a read only memory or a random access memory or both. The essential elements of a computer are a processor for performing instructions and one or more memory devices for storing instructions and data. Generally, a computer will also include, or be operatively coupled to receive data from or transfer data to, or both, one or more mass storage devices for storing data, e.g., magnetic, magneto optical disks, or optical disks. However, a computer need not have such devices. Computer readable media suitable for storing computer program instructions and data include all forms of nonvolatile memory, media and memory devices, including by way of example semiconductor memory devices, e.g., EPROM, EEPROM, and flash memory devices. The processor and the memory can be supplemented by, or incorporated in, special purpose logic circuitry.

It is intended that the specification, together with the drawings, be considered exemplary only, where exemplary means an example. As used herein, the singular forms “a”, “an” and “the” are intended to include the plural forms as well, unless the context clearly indicates otherwise. Additionally, the use of “or” is intended to include “and/or”, unless the context clearly indicates otherwise.

While this patent document contains many specifics, these should not be construed as limitations on the scope of any invention or of what may be claimed, but rather as descriptions of features that may be specific to particular embodiments of particular inventions. Certain features that are described in this patent document in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. Moreover, the separation of various system components in the embodiments described in this patent document should not be understood as requiring such separation in all embodiments.

Only a few implementations and examples are described and other implementations, enhancements and variations can be made based on what is described and illustrated in this patent document.

1. A sensor device for simultaneous monitoring two or more analytes, comprising:
a substrate; and
two or more detecting electrodes disposed on the substrate, the two or more detecting electrodes including a first electrode to sense a first analyte by an enzymatic or direct electrochemical detection, and a second electrode to sense a second analyte by an immunosensing detection;
a first functionalization layer disposed on the first electrode, the first functionalization layer including a catalyst to facilitate an electrochemical reaction to detect the first analyte at the first electrode;
a second functionalization layer disposed on the second electrode, the second functionalization layer including a capture antibody to facilitate an electrochemical immune assay reaction to detect the second analyte at the second electrode; and
a counter electrode to detect a signal with respect to that detected by the one or both of the first electrode and the second electrode.

2. The device of claim 1, wherein the first analyte includes glucose and the second analyte includes insulin.

3. The device of claim 1, wherein the first analyte includes cortisol and the second analyte includes insulin.

4. The device of claim 1, wherein the first analyte and the second analyte are contained in a first fluid or are separately contained in the first fluid and a second fluid, wherein the first fluid and the second fluid include one of saliva, blood, tears, interstitial fluid, or a combination thereof.

5. The device of claim 1, wherein the capture antibody is attached to the second electrode via a self-assembled monolayer (SAM).

6. The device of claim 1, wherein the second analyte includes insulin and the capture antibody includes an anti-insulin antibody, and wherein the second functionalization layer is configured to facilitate a sandwich electrochemical immunoassay to detect a concentration of insulin in a biofluid.

7. The device of claim 6, wherein the sandwich electrochemical immunoassay includes horseradish peroxidase (HRP) attached to a detection antibody used as an enzyme label and 3,3′,5,5′-tetramethylbenzidine (TMB)/hydrogen peroxide (H₂O₂) used as an enzymatic substrate/mediator for detection of the insulin after covalent attachment of the insulin to the capture antibody and subsequent binding of the detection antibody.

8. The device of claim 7, wherein the concentration of the insulin is detectable within a picomolar concentration range by the sensor device.

9. The device of claim 1, wherein the catalyst includes glucose oxidase (GOx).

10. The device of claim 9, wherein the GOx is immobilized inside a permeable polymer film.

11. The device of claim 10, wherein the permeable polymer film comprises a tetrathiafulvalene (TTF) mediator layer formed on the first electrode and a layer comprising GOx, chitosan, and a sulfonated tetrafluoroethylene polymer.

12. The device of claim 1, wherein the first analyte and the second analyte each include one diabetic biomarker selected from a group consisting of glucose, lactate, ketone bodies, insulin, cortisol, and C-peptide.

13. The device of claim 1, comprising:
an electronics unit including an electric circuit coupled to a data processing unit, wherein, when the sensor device is electrically coupled to the electronics unit, the device is operable to detect the first analyte and the second analyte and determine a concentration of the first analyte and of the second analyte.

14. The device of claim 1, wherein the first electrode and second electrodes include gold.

15. The device of claim 1, wherein the counter electrode includes silver/silver chloride (Ag/AgCl).

16. The device of claim 1, wherein the first electrode and the second electrode are configured on opposing sides of the counter electrode.

17. The device of claim 1, wherein the two or more detecting electrodes further include a third electrode to sense a third analyte by the enzymatic or direct electrochemical detection or by the immunosensing detection.

18. The device of claim 1, wherein the substrate includes a plastic.

19. The device of claim 18, wherein the plastic includes polyethylene terephthalate (PET), polyethylene terephthalate glycol (PETG), polyethylene naphthalate (PEN), or polyimide (PI).

20. The device of claim 1, wherein the substrate is flexible, bendable and/or stretchable.

21.-30. (canceled)
